(19) United States
(12) Patent Application Publication
(10) Pub. No.: US 2021/0386741 A1
(43) Pub. Date: Dec. 16, 2021

(54) FORMULATIONS OF IBRUTINIB

(71) Applicant: Zhuhai Beihai Biotech Co., Ltd., Zhuhai (CN)

(72) Inventor: Qun Sun, Princeton, NJ (US)

(21) Appl. No.: 16/326,014

(22) PCT Filed: Aug. 18, 2017
(86) PCT No.: PCT/US2017/047501

§ 371 Date: Feb. 15, 2019

Related U.S. Application Data
(60) Provisional application No. 62/377,333, filed on Aug. 19, 2016.

(51) Int. Cl. A61K 31/519; A61K 47/64; A61K 9/08

(52) U.S. Cl. CPC A61K 31/519; A61K 9/08; A61K 47/643

(57) ABSTRACT

This document relates to compositions comprising a non-covalently bound complex comprising ibrutinib and human serum albumin, wherein the ibrutinib and the human serum albumin in the composition have a ratio by weight from about 1:5 to about 1:2000. This document also relates to compositions comprising ibrutinib and human serum albumin, wherein the ibrutinib and the human serum albumin in the composition have a ratio by weight from about 1:5 to about 1:2000. This document also relates to compositions consisting essentially of ibrutinib and human serum albumin, wherein the ibrutinib and the human serum albumin in the composition have a ratio by weight from about 1:5 to about 1:2000.

### CLAIM OF PRIORITY

This application claims the benefit of U.S. provisional application No. 62/377,333 filed Aug. 19, 2016. The entire content of the foregoing is hereby incorporated by reference.

### TECHNICAL FIELD

This document relates to compositions and formulations for the treatment of proliferative diseases, and more particularly to compositions and formulations comprising ibrutinib.

### BACKGROUND

Ibrutinib is an anticancer drug targeting B-cell malignancies. It is an orally-administered, selective and covalent inhibitor of the enzyme Bruton's tyrosine kinase (BTK) (Pan Z et al., ChemMelBhem 2007; 2(1):58-61).

Ibrutinib (marketed under the name Imbruvica) is approved by the US Food and Drug administration (FDA) and indicated for the treatment of patients with Mantle cell lymphoma (MCL), Chronic lymphocytic leukemia (CLL)/Small lymphocytic lymphoma (SLL), Chronic lymphocytic leukemia (CLL)/Small lymphocytic lymphoma (SLL) with 17p deletion, and Waldenstrom's macroglobulinemia (WM).

Ibrutinib is a weak base and practically insoluble in water. Ibrutinib is administered orally at doses (420 mg/day or 560 mg/day) to patients. The bioavailability of ibrutinib is very low. Absolute bioavailability in fasted condition (n=8) was 2.9% (90% CI=2.1−3.9) and doubled when combined with a meal. Administration with food increased ibrutinib Cₘₐₓ and AUC by approximately 2 to 4- and 2-fold, respectively, compared with administration of ibrutinib after overnight fasting. See Imbruvica Prescribing Information. The food effects and low bioavailability result in more variable absorption and potential variability of the desired therapeutic response to patients. The lack of a suitable IV formulation has prevented the development of optimal clinical administration schedules of ibrutinib.

Accordingly, there is a need in the art for suitable IV formulations of ibrutinib. The compositions and methods described in the present application help meet this need.

### SUMMARY

Provided herein is a composition comprising a non-covalently bound complex comprising ibrutinib and human serum albumin, wherein the ibrutinib and the human serum albumin in the composition have a ratio by weight from about 1:5 to about 1:2000.

In some embodiments, the ibrutinib and the human serum albumin in the composition have a ratio by weight from about 1:50 to about 1:2000, from about 1:100 to about 1:1000, from about 1:120 to about 1:800, from about 1:130 to about 1:700, from about 1:140 to about 1:600, from about 1:150 to about 1:500, from about 1:160 to about 1:400, from about 1:170 to about 1:350, or from about 1:180 to about 1:300. In some embodiments, the ibrutinib and the human serum albumin in the composition are in a ratio by weight from about 1:140 to about 1:400. In some embodiments, the ibrutinib and the human serum albumin have a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

Also, provided herein is a composition comprising ibrutinib and human serum albumin, wherein the ibrutinib and the human serum albumin in the composition have a ratio by weight from about 1:5 to about 1:2000.

In some embodiments, the ibrutinib and the human serum albumin in the composition are in a ratio by weight from about 1:50 to about 1:2000. In some embodiments, the ibrutinib and the human serum albumin in the composition have a ratio by weight from about 1:100 to about 1:1000, from about 1:120 to about 1:800, from about 1:130 to about 1:700, from about 1:140 to about 1:600, from about 1:150 to about 1:500, from about 1:160 to about 1:400, from about 1:170 to about 1:350, or from about 1:180 to about 1:300. In some embodiments, the ibrutinib and the human serum albumin in the composition are in a ratio by weight from about 1:140 to about 1:400. In some embodiments, the ibrutinib and the human serum albumin have a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

In some embodiments, the composition is a clear aqueous solution for at least about 1 hour, 2 hours, 3 hours, 4 hours, 5 hours, 6 hours, 8 hours, or 24 hours, when the composition is dissolved in an aqueous solution.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water. In some embodiments, the aqueous formulation is a clear aqueous solution for at least 1 hour. In some embodiments, the aqueous formulation is a clear aqueous solution for at least 2 hours. In some embodiments, the aqueous formulation is a clear aqueous solution for at least 3 hours. In some embodiments, the aqueous formulation is a clear aqueous solution for at least 4 hours. In some embodiments, the aqueous formulation is a clear aqueous solution for at least 5 hours. In some embodiments, the aqueous formulation is a clear aqueous solution for at least 6 hours. In some embodiments, the aqueous formulation is a clear aqueous solution for at least 8 hours. In some embodiments, the aqueous formulation is a clear aqueous solution for at least 24 hours. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week.

Also, provided herein is a pharmaceutical composition comprising the composition comprising the ibrutinib and the human serum albumin as described herein, and a pharmaceutically acceptable carrier.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method of treating a cancer, the method comprising the step of administering to a subject in need thereof of a therapeutically effective amount of a pharmaceutical composition comprising the composition comprising the ibrutinib and the human serum albumin as described herein, and a pharmaceutically acceptable carrier.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

Also, provided herein is a composition consisting essentially of ibrutinib and human serum albumin, wherein the ibrutinib and the human serum albumin in the composition have a ratio by weight from about 1:5 to about 1:2000.

In some embodiments, the ibrutinib and the human serum albumin in the composition are in a ratio by weight from about 1:50 to about 1:2000. In some embodiments, the ibrutinib and the human serum albumin have a ratio by weight from about 1:100 to about 1:1000, from about 1:120 to about 1:800, from about 1:130 to about 1:700, from about 1:140 to about 1:600, from about 1:150 to about 1:500, from about 1:160 to about 1:400, from about 1:170 to about 1:350, or from about 1:180 to about 1:300. In some embodiments, the ibrutinib and the human serum albumin in the composition are in a ratio by weight from about 1:140 to about 1:400. In some embodiments, the ibrutinib and the human serum albumin have a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

In some embodiments, the composition is a clear aqueous solution for at least about 1 hour, 2 hours, 3 hours, 4 hours, 5 hours, 6 hours, 8 hours, or 24 hours, when the composition is dissolved in an aqueous solution.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water. In some embodiments, the aqueous formulation is a clear aqueous solution for at least 1 hour. In some embodiments, the aqueous formulation is a clear aqueous solution for at least 2 hours. In some embodiments, the aqueous formulation is a clear aqueous solution for at least 3 hours. In some embodiments, the aqueous formulation is a clear aqueous solution for at least 4 hours. In some embodiments, the aqueous formulation is a clear aqueous solution for at least 5 hours. In some embodiments, the aqueous formulation is a clear aqueous solution for at least 6 hours. In some embodiments, the aqueous formulation is a clear aqueous solution for at least 8 hours. In some embodiments, the aqueous formulation is a clear aqueous solution for at least 24 hours. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week.

Also, provided herein is a pharmaceutical composition comprising the composition comprising the ibrutinib and the human serum albumin as described herein, and a pharmaceutically acceptable carrier.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method of treating a cancer, the method comprising the step of administering to a subject in need thereof of a therapeutically effective amount of a pharmaceutical composition comprising the composition comprising the ibrutinib and the human serum albumin as described herein, and a pharmaceutically acceptable carrier.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

Also, provided herein is a composition comprising a non-covalently bound complex consisting essentially of ibrutinib and human serum albumin, wherein the ibrutinib and the human serum albumin in the composition are in a ratio by weight from about 1:5 to about 1:2000.

Also provided herein is a composition comprising ibrutinib and human serum albumin, wherein the ratio by weight of ibrutinib and the human serum albumin in the composition is from about 1:5 to about 1:2000, produced by a method comprising the steps of:

(i) obtaining an organic solution of ibrutinib in a polar water-miscible organic solvent;

(ii) obtaining a first aqueous solution of human serum albumin; and

(iii) mixing the organic solution of ibrutinib and the first aqueous solution of human serum albumin to obtain a second aqueous solution comprising the composition comprising ibrutinib and human serum albumin.

In some embodiments, the present disclosure provides a composition consisting essentially of ibrutinib and human serum albumin, wherein the ibrutinib and the human serum albumin in the composition have a ratio by weight from about 1:5 to about 1:2000, produced by a method comprising the steps of:

(i) obtaining an organic solution of ibrutinib in a polar water-miscible organic solvent;

(ii) obtaining a first aqueous solution of human serum albumin; and

(iii) mixing the organic solution of ibrutinib and the first aqueous solution of human serum albumin to obtain a second aqueous solution comprising the composition comprising ibrutinib and human serum albumin.

In some embodiments, the human serum albumin is essentially fatty acid free.

In some embodiments, the composition comprises a non-covalently bound complex comprising ibrutinib and human serum albumin.

In some embodiments, the amount of aqueous solvent in the first aqueous solution is from about 0.01 mL to about 0.05 mL per 1 mg of human serum albumin.

In some embodiments, the polar water-miscible organic solvent is an alcohol selected from the group consisting of methanol, ethanol, isopropanol, n-butanol, and mixtures thereof.

In some embodiments, the polar water-miscible organic solvent is selected from methanol, ethanol, and mixtures thereof.

In some embodiments, the aqueous solvent is water.

In some embodiments, the mixing comprises adding the organic solution to the first aqueous solution.

In some embodiments, wherein the mixing comprises adding the first aqueous solution to the organic solution.

In some embodiments, the mixing is carried out at a temperature from about 0° C. to about 25° C.

In some embodiments, the mixing is carried out at a temperature from about 0° C. to about 5° C.

In some embodiments, the mixing is carried out at about 0° C.

In some embodiments, the composition further comprises removing the polar water-miscible organic solvent from the second aqueous solution to obtain a third aqueous solution comprising the composition comprising ibrutinib and human serum albumin. In some embodiments, the composition comprises removing aqueous solvent from the third aqueous solution to obtain the composition comprising ibrutinib and human serum albumin.

In some embodiments, the composition further comprises removing the organic solvent and the aqueous solvent from the second aqueous solution to obtain the composition comprising ibrutinib and human serum albumin.

In some embodiments, the removing as carried out in vacuum.

In some embodiments, the removing is carried out by lyophilization.

In some embodiments, the composition forms a clear aqueous solution when the composition is dissolved in an aqueous solvent, and wherein the solubility of the composition in the aqueous solution is at least 10 mg/ml.

In some embodiments, the composition is a solid formulation

In some embodiments, the composition is an aqueous formulation.

In some embodiments, the aqueous formulation is substantially free of solvent other than water.

In some embodiments, the aqueous formulation is free of a surfactant.

In some embodiments, the surfactant is selected from the group consisting of CREMOPHOR® surfactants and Polysorbate 80.

In some embodiments, the aqueous formulation is a clear aqueous solution.

In some embodiments, the aqueous formulation is a clear aqueous solution for at least 1 hour, at least 2 hours, at least 3 hours, at least 4 hours, at least 5 hours, at least 6 hours, at least 8 hours, or at least 24 hours.

In some embodiments, the present disclosure provides a pharmaceutical composition comprising the composition as prepared by a process as described herein, and a pharmaceutically acceptable carrier.

In some embodiments, the present disclosure provides a method of treating a cancer, the method comprising the step of administering to a subject in need thereof a therapeutically effective amount of the pharmaceutical composition as described herein.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

### DETAILED DESCRIPTION

Provided herein is a composition comprising a non-covalently bound complex comprising ibrutinib and human serum albumin, wherein the ibrutinib and the human serum albumin in the composition have a ratio by weight from about 1:5 to about 1:2000.

In some embodiments, the present disclosure provides a composition comprising a non-covalently bound complex comprising ibrutinib and human serum albumin, wherein the ratio by weight of ibrutinib and the human serum albumin in the complex is from about 1:5 to about 1:2000.

In some embodiments, the ibrutinib and the human serum albumin in the composition have a ratio by weight from about 1:50 to about 1:2000, from about 1:100 to about 1:1000, from about 1:120 to about 1:800, from about 1:130 to about 1:700, from about 1:140 to about 1:600, from about 1:150 to about 1:500, from about 1:160 to about 1:400, from about 1:170 to about 1:350, or from about 1:180 to about 1:300. In some embodiments, the ibrutinib and the human serum albumin have a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the non-covalent interaction between ibrutinib and human serum albumin in the complex comprises hydrogen bonding. In some embodiments, the non-covalent interaction between ibrutinib and human serum albumin in the complex comprises electrostatic interaction. In some embodiments, the non-covalent interaction between ibrutinib and human serum albumin in the complex comprises hydrophobic interaction. In some embodiments, the non-covalent interaction between ibrutinib and human serum albumin in the complex comprises Van der Waals forces.

As used herein, the term “human serum albumin” refers to native and recombinant human serum albumin. Native human serum albumin and other plasma proteins can be precipitated from human plasma by varying the pH and adding ethanol, in what is known as the Cohn fractionation process (Cohn E J et al., J. Am. Chem. Soc. 1946; 68:459-475). By controlling the pH and ethanol content, semi-purified fractions of plasma proteins can be produced. One of the last proteins to precipitate in the Cohn process is native human serum albumin. After precipitation, a wet paste of crude native human serum albumin is obtained. Subsequent bioprocessing steps (purification, filtration, pasteurization, etc.) can be used to produce a purified, stabilized form of native human serum albumin for commercial use (Lin J J et al., Pharmaceutical Research 2000; 17:391-6). Recombinant human serum albumin is a highly purified animal-, virus-, and prion-free product as alternative to native human serum albumin, to which it is structurally equivalent (Bosse D et al., J. Clin. Pharmacol. 2005; 45:57-67). Recombinant human serum albumin has been produced by various hosts, both prokaryotic and eukaryotic (Chen Z et al., Biochimica et Biophysica Acta 2013; 1830:5515-5525). A fatty acid free human serum albumin can be prepared by treatment of human serum albumin with charcoal at low pH. Likewise, treatment of human serum albumin with charcoal at low pH can be used to remove fatty acids from human serum albumin (Chen R F, J. Biol. Chem. 1967; 242:173-181).

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)).

Human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

As used herein, the term “non-covalently bound complex” refers to a complex in which the bonds between the components of the complex are non-covalent bonds (e.g., weak bonds such as hydrogen bonds, electrostatic effects, n-effects, hydrophobic effects and Van der Waals forces). Further, human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Additionally, after the drug molecule binds to HSA, the drug molecule and HSA form a non-covalently bound drug and protein complex through the binding sites of HSA. This concept is commonly understood by one of ordinary skill in the art to which this disclosure belongs. One example of a non-covalently bound complex is a non-covalently bound complex of HSA and fatty acids, in which the fatty acids bind to HSA through HSA's multiple binding sites.

As used herein, the term “stable” refers to non-covalently bound complexes that do not readily disassociate and aggregate into their separate parts, e.g., do not readily dissociate and aggregate for a period of time of greater than 6 hours, 12 hours, 24 hours, or 3 days). For example, a solution including stable non-covalently bound complexes will often appear transparent whereas a solution including unstable non-covalently bound complexes will appear translucent or cloudy. Further, it will be appreciated by those of ordinary skill in the art, that after a period of time, stable non-covalently bound complexes can disassociate and aggregate into their separate parts. Thus, a solution including stable non-covalently bound complexes can become translucent or cloudy after a period of time (e.g., 6 hours, 12 hours, 24 hours, or 3 days).

In vitro, reversible binding of ibrutinib to human plasma protein was 97.3% with no concentration dependence in the range of 50 to 1000 ng/mL. See Imbruvica Prescribing Information.

As used herein, the term “essentially fatty acid free” refers to proteins (e.g. serum albumin) that contain less than about 0.02% fatty acid by weight. For example, human serum albumin that is essentially fatty acid free can contain less than 0.02% fatty acid by weight.

As used herein, the term “fatty acids” refers to non-esterified fatty acids (e.g. linoleic acid, α-linoleic acid, γ-linoleic acid).

As used herein the term ibrutinib is a compound that has the CAS No. 936563-96-1 and the following chemical structure:

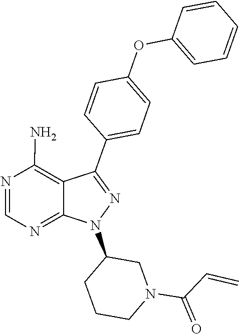 [SMILES: C=CC(=O)N1CCC[C@@H](n2nc(-c3ccc(Oc4ccccc4)cc3)c3c(N)ncnc32)C1]

Ibrutinib is a weak base and practically insoluble in water.

Further, ibrutinib is a kinase inhibitor indicated for the treatment of patients with Mantle cell lymphoma (MCL), Chronic lymphocytic leukemia (CLL)/Small lymphocytic lymphoma (SLL), Chronic lymphocytic leukemia (CLL)/Small lymphocytic lymphoma (SLL) with 17p deletion, and Waldenstrom's macroglobulinemia (WM).

In some embodiments, the ibrutinib can be a pharmaceutically acceptable salt of ibrutinib. As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, hydrogen bisulfide, bitartrate, gluconate, glucuronate, para-bromophenylsulfonate, carbonate, pyrosulfate, sulfite, bisulfate, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxybutyrate, glycolate, propanesulfonate, naphthalene-1-sulfonate, naphthalene-2-sulfonate and 2,5-dihydroxybenzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the pharmaceutically acceptable salt of ibrutinib is ibrutinib sulfate (e.g., as described in WO2016127960 or WO2016050422)

In some embodiments, ibrutinib is crystalline. In some embodiments, ibrutinib is any one of the crystalline forms disclosed, for example, in WO2016025720, WO2016127960, WO2015145415 or WO2015081180 the disclosure of which is incorporated herein by reference in its entirety.

In some embodiments, ibrutinib may be prepared by any method generally known in the art of organic synthesis. For example, ibrutinib may be prepared by a methods described in, e.g., WO2016127915, WO2016088074, WO2016079693 or WO2015074464.

In some embodiments, ibrutinib in amorphous. In some embodiments, ibrutinib is any one of the forms disclosed in, e.g., WO2016025720, WO2016127960, WO2015145415, WO2015081180, WO2016127960 or WO2016050422.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

In some embodiments, the composition is a clear aqueous solution for at least about 1 hour, 2 hours, 3 hours, 4 hours, 5 hours, 6 hours, 8 hours, or 24 hours, when the composition is dissolved in an aqueous solution.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water. In some embodiments, the aqueous formulation is a clear aqueous solution for at least 1 hour. In some embodiments, the aqueous formulation is a clear aqueous solution for at least 2 hours. In some embodiments, the aqueous formulation is a clear aqueous solution for at least 3 hours. In some embodiments, the aqueous formulation is a clear aqueous solution for at least 4 hours. In some embodiments, the aqueous formulation is a clear aqueous solution for at least 5 hours. In some embodiments, the aqueous formulation is a clear aqueous solution for at least 6 hours. In some embodiments, the aqueous formulation is a clear aqueous solution for at least 8 hours. In some embodiments, the aqueous formulation is a clear aqueous solution for at least 24 hours. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week.

Also, provided herein is a pharmaceutical composition comprising the composition comprising the ibrutinib and the human serum albumin as described herein, and a pharmaceutically acceptable carrier.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method of treating a cancer, the method comprising the step of administering to a subject in need thereof of a therapeutically effective amount of a pharmaceutical composition comprising the composition comprising the ibrutinib and the human serum albumin as described herein, and a pharmaceutically acceptable carrier.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

Also, provided herein is a composition comprising ibrutinib and human serum albumin, wherein the ibrutinib and the human serum albumin in the composition have a ratio by weight from about 1:5 to about 1:2000.

In some embodiments, the ibrutinib and the human serum albumin in the composition are in a ratio by weight from about 1:50 to about 1:2000. In some embodiments, the ibrutinib and the human serum albumin in the composition are in a ratio by weight from about 1:100 to about 1:1000. In some embodiments, the ibrutinib and the human serum albumin in the composition are in a ratio by weight from about 1:120 to about 1:800. In some embodiments, the ibrutinib and the human serum albumin in the composition are in a ratio by weight from about 1:130 to about 1:700. In some embodiments, the ibrutinib and the human serum albumin in the composition are in a ratio by weight from about 1:140 to about 1:600. In some embodiments, the ibrutinib and the human serum albumin in the composition are in a ratio by weight from about 1:150 to about 1:500. In some embodiments, the ibrutinib and the human serum albumin in the composition are in a ratio by weight from about 1:160 to about 1:400. In some embodiments, the ibrutinib and the human serum albumin in the composition are in a ratio by weight from about 1:170 to about 1:350. In some embodiments, the ibrutinib and the human serum albumin in the composition are in a ratio by weight from about 1:180 to about 1:300. In some embodiments, the ibrutinib and the human serum albumin in the composition are in a ratio by weight from about 1:130 to about 1:1000. In some embodiments, the ibrutinib and the human serum albumin in the composition are in a ratio by weight from about 1:140 to about 1:1000. In some embodiments, the ibrutinib and the human serum albumin in the composition are in a ratio by weight from about 1:150 to about 1:1000. In some embodiments, the ibrutinib and the human serum albumin in the composition are in a ratio by weight from about 1:160 to about 1:1000. In some embodiments, the ibrutinib and the human serum albumin in the composition are in a ratio by weight from about 1:170 to about 1:1000. In some embodiments, the ibrutinib and the human serum albumin in the composition are in a ratio by weight from about 1:180 to about 1:1000. In some embodiments, the ibrutinib and the human serum albumin in the composition are in a ratio by weight from about 1:140 to about 1:400. In some embodiments, the ibrutinib and the human serum albumin in the composition are in a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

In some embodiments, the ibrutinib can be a pharmaceutically acceptable salt of ibrutinib. In some embodiments, ibrutinib can be any one of crystal forms, amorphous forms, solvates and hydrates as described herein.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent.

As used herein, the term “aqueous solvent” refer to a liquid comprising at least 50%, at least 60%, at least 70%, at least 90% or at least 95% water. In some embodiments, aqueous solvent is water.

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent.

As used herein, the term “clear aqueous solution” refers to a solution containing ibrutinib and HSA in a water containing solution that is transparent upon visual observation and essentially free of visible particles or precipitation of undissolved ibrutinib.

The term “essentially free of visible particles or precipitation of undissolved ibrutinib” can be assessed as follows: after a clear aqueous solution is filtered with a 0.22 micron filter, the amount of ibrutinib in the filtered aqueous solution is at least 95% of the total amount of ibrutinib in the aqueous solution before filtration. The total amount of ibrutinib in the aqueous solution before filtration includes the particles or precipitation of undissolved ibrutinib in the aqueous solution or with the aqueous solution. The amount of the ibrutinib in an aqueous solution can be measured by the methods using HPLC. The methods of measuring the amount of the ibrutinib in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 96% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 97% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 98% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 99% of the total amount of ibrutinib in the aqueous solution before the filtration. In some embodiments, the aqueous formulation is substantially free of solvent other than water.

In some embodiments, the composition is an aqueous solution, wherein after the aqueous solution is filtered by a 0.22 micron filter, the amount of ibrutinib in the filtered aqueous solution is at least 80% of the total amount of ibrutinib in the aqueous solution before the filtration. In some embodiments, the composition is an aqueous solution, wherein after the aqueous solution is filtered by a 0.22 micron filter, the amount of ibrutinib in the filtered aqueous solution is at least 85% of the total amount of ibrutinib in the aqueous solution before the filtration. In some embodiments, the composition is an aqueous solution, wherein after the aqueous solution is filtered by a 0.22 micron filter, the amount of ibrutinib in the filtered aqueous solution is at least 90% of the total amount of ibrutinib in the aqueous solution before the filtration. In some embodiments, the aqueous formulation is substantially free of solvent other than water.

In some embodiments, the composition is a clear aqueous solution for at least 1 hour when the composition is dissolved in an aqueous solution. In some embodiments, the composition is a clear aqueous solution for at least 2 hours when the composition is dissolved in an aqueous solution. In some embodiments, the composition is a clear aqueous solution for at least 3 hours when the composition is dissolved in an aqueous solution. In some embodiments, the composition is a clear aqueous solution for at least 4 hours when the composition is dissolved in an aqueous solution. In some embodiments, the composition is a clear aqueous solution for at least 5 hours when the composition is dissolved in an aqueous solution. In some embodiments, the composition is a clear aqueous solution for at least 6 hours when the composition is dissolved in an aqueous solution. In some embodiments, the composition is a clear aqueous solution for at least 8 hours when the composition is dissolved in an aqueous solution. In some embodiments, the composition is a clear aqueous solution for at least 24 hours when the composition is dissolved in an aqueous solution. In some embodiments, the aqueous solution is substantially free of solvent other than water. In some embodiments, the aqueous solution free of solvent other than water.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation can be free of a surfactant, such as CREMOPHOR® surfactants and Polysorbate 80. In some embodiments, the aqueous formulation can be substantially free of a surfactant, such as CREMOPHOR® surfactants and Polysorbate 80. In some embodiments, the aqueous formulation can be substantially free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of surfactants and/or less than 0.0005%, less than 0.0003%, or less than 0.0001% of surfactant.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99.9% at the time of preparation, at least 99.2% after 1 hour, at least 98.1% after 2 hours, at least 97.5% after 3 hours, at least 96.7% after 4 hours, at least 95.3% after 5 hours, at least 94.4% after 6 hours, or at least 80.2% after 24 hours of the amount of ibrutinib used to prepare the composition.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99.9% at the time of preparation, at least 98.5% after 1 hour, at least 97.7% after 2 hours, at least 96.7% after 3 hours, at least 96.1% after 4 hours, at least 95.6% after 5 hours, at least 95.0% after 6 hours, or at least 82.9% after 24 hours of the amount of ibrutinib used to prepare the composition.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99% at the time of preparation, at least 98% after 1 hour, at least 97% after 2 hours, at least 96% after 3 hours, at least 96% after 4 hours, at least 95% after 5 hours, at least 94% after 6 hours, or at least 80% after 24 hours of the amount of ibrutinib used to prepare the composition.

In some embodiments, the aqueous formulation is a clear aqueous solution for at least 1 hour. In some embodiments, the aqueous formulation is a clear aqueous solution for at least 1 hour at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 2 hours. In some embodiments, the aqueous formulation is a clear aqueous solution for at least 2 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 hours. In some embodiments, the aqueous formulation is a clear aqueous solution for at least 3 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 6 hours. In some embodiments, the aqueous formulation is a clear aqueous solution for at least 6 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 24 hours. In some embodiments, the aqueous formulation is a clear aqueous solution for at least 24 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 days. In some embodiments, the aqueous formulation is a clear aqueous solution for at least 3 days when dissolved in an aqueous solution at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is substantially free of solvent other than water. In some embodiments, the aqueous formulation is free of solvent other than water.

Also, provided herein is a pharmaceutical composition comprising the composition comprising the ibrutinib and the human serum albumin as described herein, and a pharmaceutically acceptable carrier.

In some embodiments, the pharmaceutical composition further comprises at least one anti-cancer drug (e.g., any one of the anti-cancer drugs as described herein).

As used herein, the term “pharmaceutically acceptable carrier” is meant any solution used to solubilize and deliver an agent to a subject. A desirable pharmaceutically acceptable carrier is saline. Other pharmaceutically acceptable carrier and their formulation are known to one skilled in the art and described, for example, in Remington's Pharmaceutical Sciences. (20^{th} edition), ed. A. Gennaro, 2003, Lippincon Williams & Wilkins.

Pharmaceutically acceptable carriers that may be used in the pharmaceutical compositions of the present application include, but are not limited to, ion exchangers, alumina, aluminum stearate, lecithin, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, colloidal silica, magnesium trisilicate, and cellulose-based substances.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Pharmaceutically acceptable carriers that may be used in the pharmaceutical compositions of the present application include, but are not limited to, ion exchangers, alumina, aluminum stearate, lecithin, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, colloidal silica, magnesium trisilicate, and cellulose-based substances.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method of treating a proliferative disease comprising the step of administering to a subject in need thereof a pharmaceutical composition comprising the composition comprising the ibrutinib and the human serum albumin as described herein, and a pharmaceutically acceptable carrier.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

As used herein, the term “proliferative disease” refers to a disease caused by excessive proliferation of cells and turnover of cellular matrix. Non-limiting examples of proliferative diseases include cancer, atherosclerosis, arthritis (e.g. rheumatoid arthritis), psoriasis, fibrosis (e.g. pulmonary fibrosis, idiopathic pulmonary fibrosis), scleroderma and cirrhosis (e.g. cirrhosis of the liver).

Also, provided herein is a method of treating a cancer (e.g., any one of cancers described herein), the method comprising the step of administering to a subject in need thereof of a therapeutically effective amount of a pharmaceutical composition comprising the composition comprising the ibrutinib and the human serum albumin as described herein, and a pharmaceutically acceptable carrier.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, mitigation, limitation, and/or, delay of the deleterious physiological manifestations, growth or metastases of neoplasms.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

In some embodiments, the method of treating cancer (e.g., any one of cancers described herein) comprises the step of administering to a subject in need thereof a therapeutically effective amount of a pharmaceutical composition comprising the composition comprising the ibrutinib and the human serum albumin as described herein, and a therapeutically effective amount of at least one inhibitor of the following kinases for the treatment of cancer: PIM, Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, 1R-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In some embodiments, the method of treating cancer (e.g. any one of cancers described herein) comprises the step of administering to a subject in need thereof a therapeutically effective amount of a pharmaceutical composition comprising the composition comprising the ibrutinib and the human serum albumin as described herein, and a therapeutically effective amount of at least one anti-cancer drug. Examples of an anti-cancer drug include aberaterone, aberaterone acetate, abarelix, aldesleukin, alemtuzumab, alitretinoin, allopurinol, altretamine, anastrozole, arsenic trioxide, asparaginase, azacitidine, bavituximab, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, enzalutamide, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

In some embodiments, a composition comprising the ibrutinib and the human serum albumin as described herein and an anti-cancer drug are administered simultaneously.

In some embodiments, a composition comprising the ibrutinib and the human serum albumin as described herein and an anti-cancer drug are administered consecutively.

The composition comprising the ibrutinib and the human serum albumin described herein can be administered to an individual, such as human, via various routes, such as parenterally, including intravenous (e.g., as an infusion), intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the composition is administrated intravenously.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of ibrutinib will be approximately those already employed in clinical therapies wherein ibrutinib is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for ibrutinib.

Also, provided herein is a composition consisting essentially of ibrutinib and human serum albumin, wherein the ibrutinib and the human serum albumin in the composition have a ratio by weight from about 1:5 to about 1:2000.

In some embodiments, the ibrutinib and the human serum albumin in the composition are in a ratio by weight from about 1:50 to about 1:2000. In some embodiments, the ibrutinib and the human serum albumin in the composition are in a ratio by weight from about 1:100 to about 1:1000. In some embodiments, the ibrutinib and the human serum albumin in the composition are in a ratio by weight from about 1:120 to about 1:800. In some embodiments, the ibrutinib and the human serum albumin in the composition are in a ratio by weight from about 1:130 to about 1:700. In some embodiments, the ibrutinib and the human serum albumin in the composition are in a ratio by weight from about 1:140 to about 1:600. In some embodiments, the ibrutinib and the human serum albumin in the composition are in a ratio by weight from about 1:150 to about 1:500. In some embodiments, the ibrutinib and the human serum albumin in the composition are in a ratio by weight from about 1:160 to about 1:400. In some embodiments, the ibrutinib and the human serum albumin in the composition are in a ratio by weight from about 1:170 to about 1:350. In some embodiments, the ibrutinib and the human serum albumin in the composition are in a ratio by weight from about 1:180 to about 1:300. In some embodiments, the ibrutinib and the human serum albumin in the composition are in a ratio by weight from about 1:130 to about 1:1000. In some embodiments, the ibrutinib and the human serum albumin in the composition are in a ratio by weight from about 1:140 to about 1:1000. In some embodiments, the ibrutinib and the human serum albumin in the composition are in a ratio by weight from about 1:150 to about 1:1000. In some embodiments, the ibrutinib and the human serum albumin in the composition are in a ratio by weight from about 1:160 to about 1:1000. In some embodiments, the ibrutinib and the human serum albumin in the composition are in a ratio by weight from about 1:170 to about 1:1000. In some embodiments, the ibrutinib and the human serum albumin in the composition are in a ratio by weight from about 1:180 to about 1:1000. In some embodiments, the ibrutinib and the human serum albumin in the composition are in a ratio by weight from about 1:140 to about 1:400. In some embodiments, the ibrutinib and the human serum albumin in the composition are in a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

In some embodiments, the ibrutinib can be a pharmaceutically acceptable salt of ibrutinib. In some embodiments, ibrutinib can be any one of crystal forms, amorphous forms, solvates and hydrates as described herein.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 96% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 97% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 98% of the total amount of ibrutinib in the aqueous solution before the filtration. In some embodiments, the composition is a clear aqueous solution when the composition is dissolved in an aqueous solution, wherein after the clear aqueous solution is filtered by a 0.22 micron filter, the amount of ibrutinib in the filtered aqueous solution is at least 99% of the total amount of ibrutinib in the aqueous solution before the filtration. In some embodiments, the aqueous formulation is substantially free of solvent other than water.

In some embodiments, the composition is an aqueous solution, wherein after the aqueous solution is filtered by a 0.22 micron filter, the amount of ibrutinib in the filtered aqueous solution is at least 80% of the total amount of ibrutinib in the aqueous solution before the filtration. In some embodiments, the composition is an aqueous solution, wherein after the aqueous solution is filtered by a 0.22 micron filter, the amount of ibrutinib in the filtered aqueous solution is at least 85% of the total amount of ibrutinib in the aqueous solution before the filtration. In some embodiments, the composition is an aqueous solution, wherein after the aqueous solution is filtered by a 0.22 micron filter, the amount of ibrutinib in the filtered aqueous solution is at least 90% of the total amount of ibrutinib in the aqueous solution before the filtration. In some embodiments, the aqueous formulation is substantially free of solvent other than water.

In some embodiments, the composition is a clear aqueous solution for at least 1 hour when the composition is dissolved in an aqueous solution. In some embodiments, the composition is a clear aqueous solution for at least 2 hours when the composition is dissolved in an aqueous solution. In some embodiments, the composition is a clear aqueous solution for at least 3 hours when the composition is dissolved in an aqueous solution. In some embodiments, the composition is a clear aqueous solution for at least 4 hours when the composition is dissolved in an aqueous solution. In some embodiments, the composition is a clear aqueous solution for at least 5 hours when the composition is dissolved in an aqueous solution. In some embodiments, the composition is a clear aqueous solution for at least 6 hours when the composition is dissolved in an aqueous solution. In some embodiments, the composition is a clear aqueous solution for at least 8 hours when the composition is dissolved in an aqueous solution. In some embodiments, the composition is a clear aqueous solution for at least 24 hours when the composition is dissolved in an aqueous solution. In some embodiments, the aqueous solution is substantially free of solvent other than water. In some embodiments, the aqueous solution free of solvent other than water.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation can be free of a surfactant, such as CREMOPHOR® surfactants and Polysorbate 80. In some embodiments, the aqueous formulation can be substantially free of a surfactant, such as CREMOPHOR® surfactants and Polysorbate 80. In some embodiments, the aqueous formulation can be substantially free of a surfactant selected from the group consisting of CREMOPHOR® surfactants and Polysorbate 80.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99.9% at the time of preparation, at least 99.2% after 1 hour, at least 98.1% after 2 hours, at least 97.5% after 3 hours, at least 96.7% after 4 hours, at least 95.3% after 5 hours, at least 94.4% after 6 hours, or at least 80.2% after 24 hours of the amount of ibrutinib used to prepare the composition.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99.9% at the time of preparation, at least 98.5% after 1 hour, at least 97.7% after 2 hours, at least 96.7% after 3 hours, at least 96.1% after 4 hours, at least 95.6% after 5 hours, at least 95.0% after 6 hours, or at least 82.9% after 24 hours of the amount of ibrutinib used to prepare the composition.

In some embodiments, when the composition comprising ibrutinib and HSA as described herein (e.g., sterile solid powder) is dissolved in an aqueous solvent (e.g., water, saline or 5% dextrose), the resultant aqueous solution, when filtered using a 0.22 micron filter, comprises at least 99% at the time of preparation, at least 98% after 1 hour, at least 97% after 2 hours, at least 96% after 3 hours, at least 96% after 4 hours, at least 95% after 5 hours, at least 94% after 6 hours, or at least 80% after 24 hours of the amount of ibrutinib used to prepare the composition.

In some embodiments, the aqueous formulation is a clear aqueous solution for at least 1 hour. In some embodiments, the aqueous formulation is a clear aqueous solution for at least 1 hour at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 2 hours. In some embodiments, the aqueous formulation is a clear aqueous solution for at least 2 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 hours. In some embodiments, the aqueous formulation is a clear aqueous solution for at least 3 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 6 hours. In some embodiments, the aqueous formulation is a clear aqueous solution for at least 6 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 24 hours. In some embodiments, the aqueous formulation is a clear aqueous solution for at least 24 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 days. In some embodiments, the aqueous formulation is a clear aqueous solution for at least 3 days when dissolved in an aqueous solution at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is substantially free of solvent other than water. In some embodiments, the aqueous formulation is free of solvent other than water.

Also, provided herein is a pharmaceutical composition comprising the composition consisting essentially of the ibrutinib and the human serum albumin as described herein, and a pharmaceutically acceptable carrier.

In some embodiments, the pharmaceutical composition further comprises at least one anti-cancer drug (e.g., any one of the anti-cancer drugs as described herein).

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method of treating a proliferative disease comprising the step of administering to a subject in need thereof a pharmaceutical composition comprising the composition consisting essentially of the ibrutinib and the human serum albumin as described herein, and a pharmaceutically acceptable carrier.

Also, provided herein is a method of treating a cancer (e.g., any one of cancers described herein), the method comprising the step of administering to a subject in need thereof of a therapeutically effective amount of a pharmaceutical composition comprising the composition consisting essentially of the ibrutinib and the human serum albumin as described herein, and a pharmaceutically acceptable carrier.

In some embodiments, the cancer is any one of cancers described herein.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

In some embodiments, the method of treating cancer (e.g., any one of cancers described herein) comprises the step of administering to a subject in need thereof a therapeutically effective amount of a pharmaceutical composition comprising the composition consisting essentially of the ibrutinib and the human serum albumin as described herein, and a therapeutically effective amount of at least one inhibitor of the kinases for the treatment of cancer described herein.

In some embodiments, the method of treating cancer (e.g. any one of cancers described herein) comprises the step of administering to a subject in need thereof a therapeutically effective amount of a pharmaceutical composition comprising the composition consisting essentially of the ibrutinib and the human serum albumin as described herein, and a therapeutically effective amount of at least one anti-cancer drug described herein.

In some embodiments, a composition consisting essentially of the ibrutinib and the human serum albumin as described herein and an anti-cancer drug are administered simultaneously.

In some embodiments, a composition consisting essentially of the ibrutinib and the human serum albumin as described herein and an anti-cancer drug are administered consecutively.

The composition consisting essentially of the ibrutinib and the human serum albumin described herein can be administered to an individual, such as human, via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the composition is administrated intravenously.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of ibrutinib will be approximately those already employed in clinical therapies wherein ibrutinib is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for ibrutinib.

Also, provided herein is a composition comprising a non-covalently bound complex consisting essentially of ibrutinib and human serum albumin, wherein the ibrutinib and the human serum albumin in the composition are in a ratio by weight from about 1:5 to about 1:2000.

In some embodiments, the ibrutinib and the human serum albumin in the composition have a ratio by weight from about 1:100 to about 1:1000, from about 1:120 to about 1:800, from about 1:130 to about 1:700, from about 1:140 to about 1:600, from about 1:150 to about 1:500, from about 1:160 to about 1:400, from about 1:170 to about 1:350, or from about 1:180 to about 1:300. In some embodiments, the ibrutinib and the human serum albumin in the composition are in a ratio by weight from about 1:140 to about 1:400.

The present invention also includes pharmaceutical kits useful, for example, in the treatment or prevention of any one of diseases or disorders referred to herein, which include one or more containers containing a pharmaceutical composition comprising a composition of ibrutinib and the human serum albumin as described herein. Such kits can further include, if desired, one or more of various conventional pharmaceutical kit components, such as, for example, containers with one or more pharmaceutically acceptable carriers (e.g., water. Saline, or 5% dextrose), additional containers, etc., as will be readily apparent to those skilled in the art. Instructions, either as inserts or as labels, indicating quantities of the components to be administered (e.g., dosage amounts as described herein), guidelines for administration, and/or guidelines for mixing the components, can also be included in the kit.

### Methods of Making

Also, provided herein are several methods to prepare a composition comprising a non-covalently bound complex comprising the ibrutinib and the human serum albumin as described herein, a composition comprising the ibrutinib and the human serum albumin as described herein, or a composition consisting essentially of the ibrutinib and the human serum albumin as described herein.

In some embodiments, the present disclosure provides a method of preparing a composition comprising a non-covalently bound complex comprising ibrutinib and human serum albumin, wherein the ibrutinib and the human serum albumin in the composition have a ratio by weight from about 1:5 to about 1:2000.

In some embodiments, the present disclosure provides a method of preparing a composition comprising ibrutinib and human serum albumin, wherein the ibrutinib and the human serum albumin in the composition have a ratio by weight from about 1:5 to about 1:2000.

In some embodiments, the present disclosure provides a method of preparing a composition consisting essentially of ibrutinib and human serum albumin, wherein the ibrutinib and the human serum albumin in the composition have a ratio by weight from about 1:5 to about 1:2000.

In some embodiments, the method comprises mixing an organic solution of ibrutinib in a polar water-miscible organic solvent and a first aqueous solution containing human serum albumin to form a second aqueous solution, wherein the second aqueous solution is a clear aqueous solution.

In some embodiments, the method further comprises removing said polar water-miscible organic solvent and water from the second aqueous solution.

In some embodiments, the method comprises the steps of:

(i) obtaining an organic solution of ibrutinib in a polar water-miscible organic solvent;

(ii) obtaining a first aqueous solution of human serum albumin; and

(iii) mixing the organic solution of ibrutinib and the first aqueous solution of human serum albumin to obtain a second aqueous solution comprising the composition comprising ibrutinib and human serum albumin as described herein.

A non-limiting embodiments of the method are as follows.

### Formation of the Organic Solution

In some embodiments, ibrutinib is dissolved in a polar organic solvent (e.g., an alcohol such as methanol, ethanol, isopropanol, and/or n-butanol; THF, CH₃CN; DMF; or mixtures thereof) to form an organic solution.

As used herein, the term “organic solution” refers to a solution wherein at least one solvent is a non-aqueous solvent and the weight % of the non-aqueous solvent in the mixture of solvents is at least 50%, at least 60%, at least 70% or at least 90%. In some embodiments, organic solution is a solution in which does not comprise water as a solvent.

In some embodiments, the terms “organic solvent” and “non-aqueous solvent” are used interchangeably and refer to a liquid comprising is at least 50%, at least 60%, at least 70%, at least 90%, or at least 95% of a solvent other than water. In some embodiments, organic solvent is polar (e.g., polar aprotic solvent such as tetrahydrofuran, ethyl acetate, acetone, dimethylformamide, acetonitrile, dimethyl sulfoxide or nitromethane; or a polar protic solvent such as an alcohol, or an acid such as formic acid or an acetic acid). In some embodiments, the organic solvent is water-miscible (i.e., can be mixed with water in all proportions) or water-immiscible (i.e., significant proportions of organic solvent/water do not form a solution).

In some embodiments, the organic solvent is polar organic solvent that is miscible in water (e.g., tetrahydrofuran, propylene glycol, propanol, methanol, ethanol, dimethyl sulfoxide, dimethylformamide, acetonitrile or acetone). In some embodiments, the polar organic solvent is an alcohol. In some embodiments, the polar organic solvent is ethanol or methanol, or mixtures thereof. In some embodiments, the polar organic solvent can be ethanol. In some embodiments, the polar organic solvent is methanol.

In some embodiments, the amount of polar organic solvent is from about 0.005 mL to about 10 mL per 1 mg of ibrutinib. In some embodiments, the amount of polar organic solvent is from about 0.01 mL to about 5 mL per 1 mg of ibrutinib. In some embodiments, the amount of polar organic solvent is from about 0.05 mL to about 5 mL per 1 mg of ibrutinib. In some embodiments, the amount of polar organic solvent is from about 0.1 mL to about 3.0 mL per 1 mg of ibrutinib. In some embodiments, the amount of polar organic solvent is from about 1 mL to about 3 mL per 1 mg of ibrutinib. In some embodiments, the amount of polar organic solvent is from about 1.3 mL to about 3 mL per 1 mg of ibrutinib. In some embodiments, the amount of polar organic solvent is about 1 mL, about 1.3 mL, about 1.4 mL, about 1.5 mL, about 1.6 mL, about 1.7 mL, about 1.8 mL, about 1.9 mL, about 2.1 mL, about 2.6 mL, or about 3 mL per 1 mg of ibrutinib. In some embodiments, the polar organic solvent is methanol and the concentration of ibrutinib in the methanolic solution is from about 0.005 mM to about 10 mM, from about 0.05 mM to about 7 mM, from about 0.1 mM to about 5 mM, or from about 0.5 mM to about 3 mM, from about 0.5 mM to about 2 mM, or from about 0.6 mM to about 2 mM. In some embodiments, the polar organic solvent is methanol and the concentration of ibrutinib in the methanolic solution is about 0.6 mM, about 0.7 mM, about 0.8 mM, about 0.9 mM, about 1 mM, about 1.1 mM, about 1.2 mM, about 1.3 mM, about 1.5 mM, about 1.6 mM, about 1.7 mM, or about 1.9 mM.

### Formation of the First Aqueous Solution

In some embodiments, a defined amount of human serum albumin is dissolved in an amount of water to form a first aqueous solution.

In some embodiments, the amount of aqueous solvent (e.g., water) to prepare the first aqueous solution is from about 1 mL to about 10000 L, from about 2 mL to about 1000 L, from about 3 mL to about 100 L, from about 4 mL to about 10 L, from about 5 mL to about 2 L, from about 6 mL to about 1 L.

In some embodiments, the amount of HSA prepare the first aqueous solution is from about 100 mg to about 1000 kg, from about 150 mg to about 1000 kg, from about 200 mg to about 100 kg, from about 300 mg to about 5 kg, from about 200 mg to about 500 g, or from about 200 mg to about 100 g.

In some embodiments, the amount of aqueous solvent in the first aqueous solution is from about 0.005 mL to about 10 mL per 1 mg of human serum albumin. In some embodiments, the amount of aqueous solvent in the first aqueous solution is from about 0.01 mL to about 5 mL per 1 mg of human serum albumin. In some embodiments, the amount of aqueous solvent in the first aqueous solution is from about 0.01 mL to about 1 mL per 1 mg of human serum albumin. In some embodiments, the amount of aqueous solvent in the first aqueous solution is from about 0.01 mL to about 0.5 mL per 1 mg of human serum albumin. In some embodiments, the amount of aqueous solvent in the first aqueous solution is from about 0.01 mL to about 0.1 mL per 1 mg of human serum albumin. In some embodiments, the amount of aqueous solvent in the first aqueous solution is from about 0.01 mL to about 0.05 mL per 1 mg of human serum albumin. In some embodiments, the amount of aqueous solvent in the first aqueous solution is from about 0.015 mL to about 0.04 mL per 1 mg of human serum albumin. In some embodiments, the amount of aqueous solvent in the first aqueous solution is about 0.007 mL, about 0.01 mL, about 0.015 mL, about 0.02 mL, about 0.025 mL, about 0.03 mL, about 0.035 mL, about 0.04 mL, about 0.045 mL, or about 0.05 mL per 1 mg of human serum albumin. In some embodiments, the amount of aqueous solvent in the first aqueous solution about 0.02 mL per 1 mg of human serum albumin. In some embodiments, the amount of aqueous solvent (e.g., water) to prepare the first aqueous solution is from about or from about 0.005 mL to about 1 mL, from about 0.015 mL to about 0.5 mL, from about 0.015 mL to about 0.2 mL, from about 0.015 mL to about 0.1 mL, or from about 0.015 mL to about 0.05 mL per 1 mg of HSA. In some embodiments, the amount of aqueous solvent (e.g., water) to prepare the first aqueous solution is about 0.01 mL, about 0.015 mL, about 0.019 mL about 0.02 mL, about 0.021 mL, about 0.022 mL, about 0.023 mL, about 0.024 mL, about 0.025 mL, about 0.026 mL, about 0.027 mL, about 0.028 mL, about 0.029 mL or about 0.03 mL per 1 mg of HSA.

In some embodiments, the preparation of the organic solution and the preparation of the first aqueous solution are performed concurrently.

In some embodiments, the preparation of the organic solution and the preparation of the first aqueous solution are performed sequentially. In some embodiments, the preparation of the organic solution is performed before the preparation of the first aqueous solution. In some embodiments, the preparation of the first aqueous solution is performed before the preparation of the organic solution.

### Formation of the Second Aqueous Solution

In some embodiments, the organic solution of ibrutinib is mixed with the first aqueous solution of human serum albumin to form a second aqueous solution. In some embodiments, the second aqueous solution is a clear aqueous solution.

In some embodiments, the volume ratio of the amount of water to the amount of the polar organic solvent is in a range from about 1:1 to about 1000:1. In some embodiments, the volume ratio of the amount of water to the amount of the polar organic solvent is in a range from about 1.5:1 to about 100:1. In some embodiments, the volume ratio of the amount of water to the amount of the polar organic solvent is in a range from about 1.5:1 to about 20:1. In some embodiments, the volume ratio of the amount of water to the amount of the polar organic solvent is in a range from about 1.5:1 to about 10:1. In some embodiments, the volume ratio of the amount of water to the amount of the polar organic solvent is in a range from about 2:1 to about 10:1. In some embodiments, the volume ratio of the amount of water to the amount of the polar organic solvent is about 1.5:1, about 2.0:1, about 2:1, about 2.2:1, about 2.3:1, about 2.4:1, about 2.5:1, about 3:1, about 4:1, about 5:1, about 6:1, about 7:1, about 8:1, about 9:1, or about 10:1.

In some embodiments, the organic solution is added to the first aqueous solution to form a second aqueous solution. In some embodiments, the organic solution is added dropwise to the first aqueous solution to form a second aqueous solution. In some embodiments, the first aqueous solution is added to the organic solution to form a second aqueous solution. In some embodiments, the mixing is performed with agitation. In some embodiments, the mixing is performed with stirring. In some embodiments, the mixing is performed with shaking.

In some embodiments, the addition is done at the temperature from about 0° C. to about 35° C. In some embodiments, the addition is done at the temperature from about 0° C. to about 25° C. In some embodiments, the addition is done at the temperature from about 0° C. to about 10° C. In some embodiments, the addition is done at the temperature from about 0° C. to about 5° C. In some embodiments, the addition is done at the temperature about 0° C. In some embodiments, the addition is done at the temperature about 5° C. In some embodiments, the addition is done at the temperature about 10° C.

In some embodiments, the time of addition is in a range from about 0.1 min to about 24 hours. In some embodiments, the time of addition is in a range from about 1 min to about 2 hour. In some embodiments, the time of addition is in a range from about 1 min to about 1 hour. In some embodiments, the time of addition is in a range from about 5 min to about 30 min.

In some embodiments, the resulting composition comprising the ibrutinib and the human serum albumin can have any ratio by weight of the ibrutinib to the human serum albumin as described herein. In some embodiments, the human serum albumin is a fatty acid free human serum albumin. In some embodiments, the human serum albumin is essentially fatty acid free.

### Removal of Organic Solvent

In some embodiments, upon completion of mixing of the organic solution with the first aqueous solution to form the second aqueous solution, the polar organic solvent is removed from the second aqueous solution.

In some embodiments, the polar organic solvent is removed under reduced pressure. In some embodiments, the polar organic solvent is removed using rotary evaporation. In some embodiments, the polar organic solvent is removed under a vacuum.

In some embodiments, the removal of the polar organic solvent yields a clear aqueous solution. In some embodiments, water is removed from the aqueous under a vacuum. In some embodiments, water is removed from the aqueous solution using rotary evaporation. In some embodiments, water is removed from the aqueous solution by lyophilization.

In some embodiments, the solvents including both water and organic solvent are removed from the second aqueous solution simultaneously to provide a solid composition. In some embodiments, the solvents are removed under a vacuum. In some embodiments, the solvents are removed using rotary evaporation. In some embodiments, the solvents are removed by lyophilization. In some embodiments, the second aqueous solution was filtered before removal of the solvents.

Removal of Water from the Second Aqueous Solution

In some embodiments, upon removal of the organic solvent from the second aqueous solution, the water can be removed from the second aqueous solution to provide a solid composition.

In some embodiments, the second aqueous solution is filtered before removal of water. For example, the second aqueous solution can be filtered by a 0.22 micron filter before removal of water.

As used herein, the term “micron” refers to a unit of measure of one one-thousandth of a millimeter.

In some embodiments, the water is removed under a vacuum. In some embodiments, the water is removed using rotary evaporation. In some embodiments, the water is removed by lyophilization.

### Reconstitution of the Solid

In some embodiments the solid comprising the ibrutinib and the human serum albumin is mixed with an aqueous solution. In some embodiments, the aqueous solution is a saline solution. In some embodiments, the aqueous solution is a 5% Dextrose aqueous solution. In some embodiments, the mixing is the addition of the aqueous solution to the solid. In some embodiments, the mixing is the addition of the solid to the aqueous solution. In some embodiments, the mixing reconstitutes the solid. In some embodiments, the mixing yields a clear aqueous solution.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

### Composition Prepared by the Process

In some embodiments, the present disclosure provides a composition comprising ibrutinib and human serum albumin, wherein the ibrutinib and the human serum albumin in the composition have a ratio by weight as described herein (e.g., from about 1:5 to about 1:2000), produced by a method comprising the steps of:

(i) obtaining an organic solution of ibrutinib in a polar water-miscible organic solvent;

(ii) obtaining a first aqueous solution of human serum albumin; and

(iii) mixing the organic solution of ibrutinib and the first aqueous solution of human serum albumin to obtain a second aqueous solution comprising the composition comprising ibrutinib and human serum albumin.

In some embodiments, the ibrutinib and the human serum albumin in the composition are in a ratio by weight from about 1:50 to about 1:2000. In some embodiments, the ibrutinib and the human serum albumin in the composition are in a ratio by weight from about 1:100 to about 1:1000. In some embodiments, the ibrutinib and the human serum albumin in the composition are in a ratio by weight from about 1:120 to about 1:800. In some embodiments, the ibrutinib and the human serum albumin in the composition are in a ratio by weight from about 1:130 to about 1:700. In some embodiments, the ibrutinib and the human serum albumin in the composition are in a ratio by weight from about 1:140 to about 1:600. In some embodiments, the ibrutinib and the human serum albumin in the composition are in a ratio by weight from about 1:150 to about 1:500. In some embodiments, the ibrutinib and the human serum albumin in the composition are in a ratio by weight from about 1:160 to about 1:400. In some embodiments, the ibrutinib and the human serum albumin in the composition are in a ratio by weight from about 1:170 to about 1:350. In some embodiments, the ibrutinib and the human serum albumin in the composition are in a ratio by weight from about 1:180 to about 1:300. In some embodiments, the ibrutinib and the human serum albumin in the composition are in a ratio by weight from about 1:130 to about 1:1000. In some embodiments, the ibrutinib and the human serum albumin in the composition are in a ratio by weight from about 1:140 to about 1:1000. In some embodiments, the ibrutinib and the human serum albumin in the composition are in a ratio by weight from about 1:150 to about 1:1000. In some embodiments, the ibrutinib and the human serum albumin in the composition are in a ratio by weight from about 1:160 to about 1:1000. In some embodiments, the ibrutinib and the human serum albumin in the composition are in a ratio by weight from about 1:170 to about 1:1000. In some embodiments, the ibrutinib and the human serum albumin in the composition are in a ratio by weight from about 1:180 to about 1:1000. In some embodiments, the ibrutinib and the human serum albumin in the composition are in a ratio by weight from about 1:140 to about 1:400. In some embodiments, the ibrutinib and the human serum albumin in the composition are in a ratio by weight of about 1:140, about 1:150, about 1:160, about 1:170, about 1:180, about 1:190, about 1:200, about 1:210, about 1:220, about 1:225, about 1:230, about 1:240, about 1:250, about 1:260, about 1:270, or about 1:280, about 1:290, about 1:300, about 1:350, or about 1:400.

In some embodiments, the present disclosure provides a composition consisting essentially of ibrutinib and human serum albumin, wherein the ibrutinib and the human serum albumin in the composition have a ratio by weight as described herein (e.g., from about 1:5 to about 1:2000), produced by a method comprising the steps of:

(i) obtaining an organic solution of ibrutinib in a polar water-miscible organic solvent;

(ii) obtaining a first aqueous solution of human serum albumin; and

(iii) mixing the organic solution of ibrutinib and the first aqueous solution of human serum albumin to obtain a second aqueous solution comprising the composition comprising ibrutinib and human serum albumin.

In some embodiments, the ibrutinib and the human serum albumin in the composition are in a ratio by weight from about 1:50 to about 1:2000. In some embodiments, the ibrutinib and the human serum albumin in the composition are in a ratio by weight from about 1:100 to about 1:1000. In some embodiments, the ibrutinib and the human serum albumin in the composition are in a ratio by weight from about 1:120 to about 1:800. In some embodiments, the ibrutinib and the human serum albumin in the composition are in a ratio by weight from about 1:130 to about 1:700. In some embodiments, the ibrutinib and the human serum albumin in the composition are in a ratio by weight from about 1:140 to about 1:600. In some embodiments, the ibrutinib and the human serum albumin in the composition are in a ratio by weight from about 1:150 to about 1:500. In some embodiments, the ibrutinib and the human serum albumin in the composition are in a ratio by weight from about 1:160 to about 1:400. In some embodiments, the ibrutinib and the human serum albumin in the composition are in a ratio by weight from about 1:170 to about 1:350. In some embodiments, the ibrutinib and the human serum albumin in the composition are in a ratio by weight from about 1:180 to about 1:300. In some embodiments, the ibrutinib and the human serum albumin in the composition are in a ratio by weight from about 1:130 to about 1:1000. In some embodiments, the ibrutinib and the human serum albumin in the composition are in a ratio by weight from about 1:140 to about 1:1000. In some embodiments, the ibrutinib and the human serum albumin in the composition are in a ratio by weight from about 1:150 to about 1:1000. In some embodiments, the ibrutinib and the human serum albumin in the composition are in a ratio by weight from about 1:160 to about 1:1000. In some embodiments, the ibrutinib and the human serum albumin in the composition are in a ratio by weight from about 1:170 to about 1:1000. In some embodiments, the ibrutinib and the human serum albumin in the composition are in a ratio by weight from about 1:180 to about 1:1000. In some embodiments, the ibrutinib and the human serum albumin in the composition are in a ratio by weight from about 1:140 to about 1:400. In some embodiments, the ibrutinib and the human serum albumin in the composition are in a ratio by weight of about 1:140, about 1:150, about 1:160, about 1:170, about 1:180, about 1:190, about 1:200, about 1:210, about 1:220, about 1:230, about 1:240, about 1:250, about 1:260, about 1:270, or about 1:280, about 1:290, about 1:300, about 1:350, or about 1:400.

In some embodiments, the ibrutinib can be a pharmaceutically acceptable salt of ibrutinib. In some embodiments, ibrutinib can be any one of crystal forms, amorphous forms, solvates and hydrates as described herein.

In some embodiments, the human serum albumin is essentially fatty acid free.

In some embodiments, the composition comprises a non-covalently bound complex comprising ibrutinib and human serum albumin.

In some embodiments, the amount of polar water-miscible organic solvent is from about 0.005 mL to about 10 mL per 1 mg of ibrutinib. In some embodiments, the amount of organic solvent is from about 0.01 mL to about 5 mL per 1 mg of ibrutinib. In some embodiments, the amount of organic solvent is from about 0.05 mL to about 5 mL per 1 mg of ibrutinib. In some embodiments, the amount of organic solvent is from about 0.1 mL to about 3 mL per 1 mg of ibrutinib. In some embodiments, the amount of polar organic solvent is from about 0.05 mL to about 5 mL per 1 mg of ibrutinib. In some embodiments, the amount of polar organic solvent is from about 0.1 mL to about 3.0 mL per 1 mg of ibrutinib. In some embodiments, the amount of polar organic solvent is from about 1 mL to about 3 mL per 1 mg of ibrutinib. In some embodiments, the amount of polar organic solvent is from about 1.3 mL to about 3 mL per 1 mg of ibrutinib. In some embodiments, the amount of polar organic solvent is about 1 mL, about 1.3 mL, about 1.4 mL, about 1.5 mL, about 1.6 mL, about 1.7 mL, about 1.8 mL, about 1.9 mL, about 2.1 mL, about 2.6 mL, or about 3 mL per 1 mg of ibrutinib. In some embodiments, the polar organic solvent is methanol and the concentration of ibrutinib in the organic solution is from about 0.005 mM to about 10 mM, from about 0.05 mM to about 7 mM, from about 0.1 mM to about 5 mM, or from about 0.5 mM to about 3 mM, from about 0.5 mM to about 2 mM, or from about 0.6 mM to about 2 mM. In some embodiments, the polar organic solvent is methanol and the concentration of ibrutinib in the organic solution is about 0.6 mM, about 0.7 mM, about 0.8 mM, about 0.9 mM, about 1 mM, about 1.1 mM, about 1.2 mM, about 1.3 mM, about 1.5 mM, about 1.6 mM, about 1.7 mM, or about 1.9 mM.

In some embodiments, the amount of aqueous solvent in the first aqueous solution is from about 0.01 mL to about 0.05 mL per 1 mg of human serum albumin.

In some embodiments, the amount of aqueous solvent in the first aqueous solution is from about 0.015 mL to about 0.04 mL or from about 0.015 mL to about 0.022 mL per 1 mg of human serum albumin.

In some embodiments, the amount of aqueous solvent in the first aqueous solution is from about 0.005 mL to about 10 mL per 1 mg of human serum albumin. In some embodiments, the amount of aqueous solvent in the first aqueous solution is from about 0.01 mL to about 5 mL per 1 mg of human serum albumin. In some embodiments, the amount of aqueous solvent in the first aqueous solution is from about 0.01 mL to about 1 mL per 1 mg of human serum albumin. In some embodiments, the amount of aqueous solvent in the first aqueous solution is from about 0.01 mL to about 0.5 mL per 1 mg of human serum albumin. In some embodiments, the amount of aqueous solvent in the first aqueous solution is from about 0.01 mL to about 0.1 mL per 1 mg of human serum albumin. In some embodiments, the amount of aqueous solvent in the first aqueous solution is from about 0.01 mL to about 0.05 mL per 1 mg of human serum albumin. In some embodiments, the amount of aqueous solvent in the first aqueous solution is from about 0.015 mL to about 0.04 mL per 1 mg of human serum albumin. In some embodiments, the amount of aqueous solvent in the first aqueous solution is about 0.007 mL, about 0.01 mL, about 0.015 mL, about 0.02 mL, about 0.025 mL, about 0.03 mL, about 0.035 mL, about 0.04 mL, about 0.045 mL, or about 0.05 mL per 1 mg of human serum albumin. In some embodiments, the amount of aqueous solvent (e.g., water) to prepare the first aqueous solution is from about or from about 0.005 mL to about 1 mL, from about 0.015 mL to about 0.5 mL, from about 0.015 mL to about 0.2 mL, from about 0.015 mL to about 0.1 mL, or from about 0.015 mL to about 0.05 mL per 1 mg of HSA. In some embodiments, the amount of aqueous solvent (e.g., water) to prepare the first aqueous solution is about 0.01 mL, about 0.015 mL, about 0.019 mL about 0.02 mL, about 0.021 mL, about 0.022 mL, about 0.023 mL, about 0.024 mL, about 0.025 mL, about 0.026 mL, about 0.027 mL, about 0.028 mL, about 0.029 mL or about 0.03 mL per 1 mg of HSA. In some embodiments, the amount of aqueous solvent in the first aqueous solution about 0.02 mL per 1 mg of human serum albumin.

In some embodiments, the polar water-miscible organic solvent is an alcohol selected from the group consisting of methanol, ethanol, isopropanol, n-butanol, and mixtures thereof.

In some embodiments, the polar water-miscible organic solvent is selected from methanol, ethanol, and mixtures thereof.

In some embodiments, the polar water-miscible organic solvent is methanol.

In some embodiments, the aqueous solvent is water.

In some embodiments, the polar water-miscible organic solvent is methanol and the aqueous solvent in the first aqueous solution is water.

In some embodiments, the mixing comprises adding the organic solution to the first aqueous solution. In some embodiments, wherein the mixing comprises adding the first aqueous solution to the organic solution. In some embodiments, the adding is carried out dropwise. In some embodiments, the adding is carried out for a period of time from several minutes to several hours. In some embodiments, the adding is carried out for a period of time from 2 min to 24 hours. In some embodiments, the adding is carried out for a period of time from 2 min minutes to 12 hours, from 2 min to 6 hours, from 3 min to 3 hours, from 2 min to 1 hour, from 2 min to 30 min, or from 2 min to 25 min.

In some embodiments, the mixing is carried out at a temperature from about 0° C. to about 25° C. In some embodiments, mixing is carried out at ambient temperature (e.g., about 25° C.). In some embodiments, the mixing is carried out at a temperature from about 0° C. to about 5° C. In some embodiments, the mixing is carried out at about 0° C.

In some embodiments, the volume ratio of the amount of aqueous solvent to the amount of the organic solvent in the second aqueous solution is in a range from about 1:1 to about 1000:1. In some embodiments, the volume ratio of the amount of aqueous solvent to the amount of the organic solvent in the second aqueous solution is in a range from about 1.5:1 to about 100:1. In some embodiments, the volume ratio of the amount of aqueous solvent to the amount of the organic solvent in the second aqueous solution is in a range from about 1.5:1 to about 20:1. In some embodiments, the volume ratio of the amount of aqueous solvent to the amount of the organic solvent in the second aqueous solution is in a range from about 1.5:1 to about 10:1. In some embodiments, the volume ratio of the amount of aqueous solvent to the amount of the organic solvent in the second aqueous solution is in a range from about 2:1 to about 10:1. In some embodiments, the volume ratio of the amount of aqueous solvent to the amount of the organic solvent in the second aqueous solution is about 1.5:1, about 2.0:1, about 2:1, about 2.2:1, about 2.3:1, about 2.4:1, about 2.5:1, about 3:1, about 4:1, about 5:1, about 6:1, about 7:1, about 8:1, about 9:1, or about 10:1. In some embodiments, the aqueous solvent is water. In some embodiments, the aqueous solvent is water and the organic solvent is an alcohol. In some embodiments, the aqueous solvent is water and the organic solvent is methanol.

In some embodiments, the composition further comprises removing the polar water-miscible organic solvent from the second aqueous solution to obtain a third aqueous solution comprising the composition comprising ibrutinib and human serum albumin. In some embodiments, the composition comprises removing aqueous solvent from the third aqueous solution to obtain the composition comprising ibrutinib and human serum albumin.

In some embodiments, the composition further comprises removing the organic solvent (e.g. methanol) and the aqueous solvent (e.g., water) from the second aqueous solution to obtain the composition comprising ibrutinib and human serum albumin.

In some embodiments, the removing as carried out in vacuum (e.g., using the rotovap). In some embodiments, the removing is carried out by lyophilization.

In some embodiments, the composition forms a clear aqueous solution when the composition is dissolved in an aqueous solvent, and wherein the solubility of the composition in the aqueous solution is at least 10 mg/ml.

In some embodiments, the composition is a solid formulation

In some embodiments, the composition is an aqueous formulation. In some embodiments, the aqueous formulation is substantially free of solvent other than water. In some embodiments, the aqueous formulation is free of a surfactant. In some embodiments, the surfactant is selected from the group consisting of CREMOPHOR® surfactants and Polysorbate 80.

In some embodiments, the aqueous formulation is a clear aqueous solution. In some embodiments, the aqueous formulation is a clear aqueous solution for at least 1 hour, at least 2 hours, at least 3 hours, at least 4 hours, at least 5 hours, at least 6 hours, at least 8 hours, or at least 24 hours.

In some embodiments, the present disclosure provides a pharmaceutical composition comprising the composition as prepared by a process as described herein, and a pharmaceutically acceptable carrier.

In some embodiments, the present disclosure provides a method of treating a cancer, the method comprising the step of administering to a subject in need thereof a therapeutically effective amount of the pharmaceutical composition as described herein.

In some embodiments, the cancer is a cancer of the blood. In some embodiments, the cancer is selected from the group consisting of Mantel cell lymphoma (MCL), Chronic lymphocytic leukemia (CLL), Small lymphocytic lymphoma (SLL), Diffuse large B cell lymphoma (DLBCL), Follicular Lymphoma (FL), and Waldenstrom's macroglobulinemia (WM). In some embodiments, the cancer is a Mantel cell lymphoma. In some embodiments, the cancer is a Chronic lymphocytic leukemia/Small lymphocytic lymphoma. In some embodiments, the cancer is a Chronic lymphocytic leukemia/Small lymphocytic lymphoma with 17p deletion. In some embodiments, the cancer is a Waldenstrom's macroglobulinemia (WM). In some embodiments the cancer is a Diffuse large B cell lymphoma. In some embodiments, the cancer is a Follicular Lymphoma.

### EXAMPLES

### Materials and Methods

HPLC analysis: The HPLC system used herein is a SHIMADZU LC-10AT vp series system, which consists of a SHIMADZU LC-10AT vp pump, a manual injector, a SHIMADZU CTO-10AS vp column oven, a SHIMADZU SPD-10A vp wavelength detector, and a SHIMADZU LC solution workstation. Waters XTERRA RP10 column (4.6 mm×150 mm, 5 μm) is used as an analytical HPLC column. The column oven temperature is 30° C. Mobile phase is composed of methanol and water (70:30, v/v) and pumped at a flow rate of 1 ml/minute. The effluent is detected at a wavelength of 233 nm using a UV detector. The sample injection amount is 20 μl.

### Example 1: Composition Comprising Ibrutinib and Human Serum Albumin (HSA)

The Ratio by Weight of Ibrutinib to HSA Prepared was about 1:200.

Ibrutinib (2 mg) was dissolved in methanol (3.5 ml) in a vial to give a clear solution. HSA (400 mg) (native fatty acid free human serum albumin purchased from SeraCare Life Sciences, product code: HS-455-80, which contains fatty acids <0.2 mg/gm) as a powder was dissolved in 8 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 3 hours at room temperature. This aqueous solution stays clear with no precipitation after 6 hours at room temperature.

### Example 2: Composition Comprising Ibrutinib and Human Serum Albumin (HSA)

The Ratio by Weight of Ibrutinib to HSA Prepared was about 1:250.

Ibrutinib (2 mg) was dissolved in methanol (4.3 ml) in a vial to give a clear solution. HSA (500 mg) (native fatty acid free human serum albumin purchased from SeraCare Life Sciences, product code: HS-455-80, which contains fatty acids <0.2 mg/gm) as a powder was dissolved in 10 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 3 hours at room temperature. This aqueous solution stays clear with no precipitation after 6 hours at room temperature.

### Example 3: Composition Comprising Ibrutinib and Human Serum Albumin (HSA)

The Ratio by Weight of Ibrutinib to HSA Prepared was about 1:220.

Ibrutinib (2 mg) was dissolved in methanol (3.8 ml) in a vial to give a clear solution. HSA (440 mg) (native fatty acid free human serum albumin purchased from SeraCare Life Sciences, product code: HS-455-80, which contains fatty acids <0.2 mg/gm) as a powder was dissolved in 8.8 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 4: Composition Comprising Ibrutinib and Human Serum Albumin (HSA)

The Ratio by Weight of Ibrutinib to HSA Prepared was about 1:225.

Ibrutinib (5 mg) was dissolved in methanol (9.6 ml) in a vial to give a clear solution. HSA (1125 mg) (native fatty acid free human serum albumin purchased from SeraCare Life Sciences, product code: HS-455-80, which contains fatty acids <0.2 mg/gm) as a powder was dissolved in 22.5 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 5: Composition Comprising Ibrutinib and Human Serum Albumin (HSA)

The Ratio by Weight of Ibrutinib to HSA Prepared was about 1:200.

Ibrutinib (2 mg) was dissolved in methanol (3.2 ml) in a vial to give a clear solution. HSA (400 mg) (native fatty acid free human serum albumin purchased from Golden West Biologicals, Inc., CAT #: HA1020) as a powder was dissolved in 8 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 6: Composition Comprising Ibrutinib and Human Serum Albumin (HSA)

The Ratio by Weight of Ibrutinib to HSA Prepared was about 1:160.

Ibrutinib (2 mg) was dissolved in methanol (3 ml) in a vial to give a clear solution. HSA (320 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. The precipitation was formed in the solution after 24 hours at room temperature.

### Example 7: Composition Comprising Ibrutinib and Human Serum Albumin (HSA)

The Ratio by Weight of Ibrutinib to HSA Prepared was about 1:180.

Ibrutinib (2 mg) was dissolved in methanol (3 ml) in a vial to give a clear solution. HSA (360 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. The precipitation was formed in the solution after 24 hours at room temperature.

### Example 8: Composition Comprising Ibrutinib and Human Serum Albumin (HSA)

The Ratio by Weight of Ibrutinib to HSA Prepared was about 1:300.

Ibrutinib (1 mg) was dissolved in methanol (3 ml) in a vial to give a clear solution. HSA (300 mg) (native fatty acid free human serum albumin purchased from SeraCare Life Sciences, product code: HS-455-80, which contains fatty acids <0.2 mg/gm) as a powder was dissolved in 6 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 24 hours at room temperature.

### Example 9: Composition Comprising Ibrutinib and Human Serum Albumin (HSA)

The Ratio by Weight of Ibrutinib to HSA Prepared was about 1:400.

Ibrutinib (1 mg) was dissolved in methanol (3.5 ml) in a vial to give a clear solution. HSA (400 mg) (native fatty acid free human serum albumin purchased from SeraCare Life Sciences, product code: HS-455-80, which contains fatty acids <0.2 mg/gm) as a powder was dissolved in 8 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 24 hours at room temperature.

### Example 10: Composition Comprising Ibrutinib and Human Serum Albumin (HSA)

The Ratio by Weight of Ibrutinib to HSA Prepared was about 1:200.

Ibrutinib (15 mg) was dissolved in methanol (19.3 ml) in a vial to give a clear solution. HSA (3000 mg) (native fatty acid free human serum albumin purchased from SeraCare Life Sciences, product code: HS-455-80, which contains fatty acids <0.2 mg/gm) as a powder was dissolved in 45 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 24 hours at room temperature.

### Example 11: Composition Comprising Ibrutinib and Human Serum Albumin (HSA)

The Ratio by Weight of Ibrutinib to HSA Prepared was about 1:200.

Ibrutinib (1.5 mg) was dissolved in methanol (2.6 ml) in a vial to give a clear solution. HSA (300 mg) (native fatty acid free human serum albumin purchased from Golden West Biologicals, Inc., CAT #: HA1020) as a powder was dissolved in 6 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 4 hours at room temperature. This aqueous solution stays clear with no precipitation after 6 hours at room temperature. The precipitation was formed in the solution after 24 hours at room temperature.

### Example 12: Composition Comprising Ibrutinib and Human Serum Albumin (HSA)

The Ratio by Weight of Ibrutinib to HSA Prepared was about 1:140.

Ibrutinib (2 mg) was dissolved in methanol (2.6 ml) in a vial to give a clear solution. HSA (280 mg) (native fatty acid free human serum albumin purchased from SeraCare Life Sciences, product code: HS-455-80, which contains fatty acids <0.2 mg/gm) as a powder was dissolved in 6 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. The precipitation was formed in the solution after 2 hours at room temperature.

### Example 13: Composition Comprising Ibrutinib and Human Serum Albumin (HSA)

The Ratio by Weight of Ibrutinib to HSA Prepared was about 1:150.

Ibrutinib (2 mg) was dissolved in methanol (2.6 ml) in a vial to give a clear solution. HSA (300 mg) (native fatty acid free human serum albumin purchased from SeraCare Life Sciences, product code: HS-455-80, which contains fatty acids <0.2 mg/gm) as a powder was dissolved in 6 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. The precipitation was formed in the solution after 2 hours at room temperature.

### Example 14: Composition Comprising Ibrutinib and Human Serum Albumin (HSA)

The Ratio by Weight of Ibrutinib to HSA Prepared was about 1:300.

Ibrutinib (1 mg) was dissolved in methanol (2.6 ml) in a vial to give a clear solution. A solution of HSA (300 mg, 1.5 ml) (20% human serum albumin solution for infusion (product name: AlbuRx) from CSL Behring) was added into 4.5 ml of water to give a HSA solution (6 ml) in a round bottom flask. The methanol solution of ibrutinib was added slowly dropwise into the flask of the HSA solution with rapid stiffing at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 4 hours at room temperature. This aqueous solution stays clear with no precipitation after 24 hours at room temperature.

### Example 15: Composition Comprising Ibrutinib and Human Serum Albumin (HSA)

The Ratio by Weight of Ibrutinib to HSA Prepared was about 1:200.

Ibrutinib (1 mg) was dissolved in methanol (1.7 ml) in a vial to give a clear solution. A solution of HSA (200 mg, 1 ml) (20% human serum albumin solution for infusion (product name: AlbuRx) from CSL Behring) was added into 3 ml of water to give a HSA solution (4 ml) in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 4 hours at room temperature. This aqueous solution stays clear with no precipitation after 6 hours at room temperature. The precipitation was formed in the solution after 24 hours at room temperature.

### Example 16: Composition Comprising Ibrutinib and Human Serum Albumin (HSA)

The Ratio by Weight of Ibrutinib to HSA Prepared was about 1:240.

Ibrutinib (1 mg) was dissolved in methanol (2.1 ml) in a vial to give a clear solution. A solution of HSA (240 mg, 1.2 ml) (20% human serum albumin solution for infusion (product name: AlbuRx) from CSL Behring) was added into 3.6 ml of water to give a HSA solution (4.8 ml) in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 4 hours at room temperature. This aqueous solution stays clear with no precipitation after 6 hours at room temperature. The precipitation was formed in the solution after 24 hours at room temperature.

### Example 17: Measure the Correlation Between HPLC Peak Area and the Ibrutinib Concentration

Methanol solutions of ibrutinib in 7 different concentrations, 0.005 mg/ml, 0.01 mg/ml, 0.025 mg/ml, 0.0375 mg/ml, 0.05 mg/ml, 0.075 mg/ml and 0.1 mg/ml, were prepared. The 7 ibrutinib methanol solutions were analyzed in HPLC. The peak area and concentration of ibrutinib were correlated using linear regression. The linear regression data is shown as below.

Y (peak area)=50109+6.84051E7*X (concentration), R=0.99996, P<0.0001.

### Example 18: Measure the Ibrutinib Concentrations in the Aqueous Solutions Before and after the Filtration at 0 Hour, and after the Filtration at 1 Hour, 2 Hour, 3 Hour, 4 Hour, 5 Hour, 6 Hour, and 24 Hour

2.5 g of the lyophilized solid of the composition comprising ibrutinib and HSA (the ratio by weight about 1:200) from Example 10 was dissolved in 50 ml of water to give a clear aqueous solution, which was kept at about 25° C. Immediately after the lyophilized solid was dissolved in water, 6 ml of the clear aqueous solution was taken out from the 50 ml solution. Then 1 ml of the solution was taken out from the 6 ml clear aqueous solution to give the solution IB-0-0h, and the remaining 5 ml of the solution was filtered by the same 0.22 micron aqueous phase filter at 1 ml at a time to give the solutions IB-1-0h, IB-2-0h, IB-3-0h, IB-4-0h, and IB-5-0h. To 200 μl of the solutions IB-0-0h and IB-5-0h were added 800 μl of acetonitrile separately. The mixtures were vortexed for seconds and then centrifuged at 4,000 g for 5 minutes. The supernatants were removed and collected followed by injection on HPLC. The same procedure was repeated 2 more times for each of solutions IB-0-0h and IB-5-0h. Based on the HPLC data and the measurement data of example 17, the ibrutinib concentrations of the solutions of IB-0-0h, and IB-5-0h have been calculated and shown in the Table 1. At 0 hour, the ibrutinib concentration of the clear aqueous solution after the filtration was about 99.91% of the ibrutinib concentration of the clear aqueous solution before the filtration.

TABLE 1

| | Ibrutinib | Average Ibrutinib |
|---|---|---|
| | Concentration | Concentration |
| Solution Number | (mg/ml) | (mg/ml) |
| IB-0-0h-1 | 0.2239 | 0.2242 |
| IB-0-0h-2 | 0.2239 | |
| IB-0-0h-3 | 0.2249 | |
| IB-5-0h-1 | 0.2241 | |
| IB-5-0h-2 | 0.2239 | 0.2240 |
| IB-5-0h-3 | 0.2241 | |

At 1 hour, 5 ml of the clear aqueous solution was taken out from the remaining 44 ml of the aqueous solution. Then 1 ml of the solution was taken out from the 5 ml clear aqueous solution and filtered by a 0.22 micron aqueous phase filter to give the solution IB-1-1h, and the remaining 4 ml of the solution was filtered by the same 0.22 micron aqueous phase filter at 1 ml at a time to give the solutions IB-2-1h, IB-3-1h, IB-4-1h, and IB-5-1h. To 200 μl of the solution IB-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution IB-5-1h. Based on the HPLC data and the measurement data of example 17, the ibrutinib concentrations of the solution IB-5-1h have been calculated and shown in the Table 2. At 1 hour, the ibrutinib concentration of the clear aqueous solution after the filtration was about 99.15% of the ibrutinib concentration of the clear aqueous solution at 0 hour before the filtration.

TABLE 2

| | | Average |
|---|---|---|
| | Ibrutinib | Ibrutinib |
| Solution | Concentration | Concentration |
| Number | (mg/ml) | (mg/ml) |
| IB-5-1h-1 | 0.2227 | 0.2223 |
| IB-5-1h-2 | 0.2226 | |
| IB-5-1h-3 | 0.2215 | |

At 2 hours, 5 ml of the clear aqueous solution was taken out from the remaining 39 ml of the aqueous solution. The experiments were done for the 5 ml of the clear aqueous solution taken out at 2 hours using the same protocol as for the 5 ml of the clear aqueous solution taken out at 1 hour. Based on the HPLC data and the measurement data of example 17, the ibrutinib concentrations of the solution IB-5-2h have been calculated and shown in the Table 3. At 2 hours, the ibrutinib concentration of the clear aqueous solution after the filtration was about 98.13% of the ibrutinib concentration of the clear aqueous solution at 0 hour before the filtration.

TABLE 3

| | | Average |
|---|---|---|
| | Ibrutinib | Ibrutinib |
| Solution | Concentration | Concentration |
| Number | (mg/ml) | (mg/ml) |
| IB-5-2h-1 | 0.2196 | 0.2200 |
| IB-5-2h-2 | 0.2202 | |
| IB-5-2h-3 | 0.2201 | |

At 3 hours, 5 ml of the clear aqueous solution was taken out from the remaining 34 ml of the aqueous solution. The experiments were done for the 5 ml of the clear aqueous solution taken out at 3 hour using the same protocol as for the 5 ml of the clear aqueous solution taken out at 1 hour. Based on the HPLC data and the measurement data of example 17, the ibrutinib concentrations of the solution IB-5-3h have been calculated and shown in the Table 4. At 3 hours, the ibrutinib concentration of the clear aqueous solution after the filtration was about 97.46% of the ibrutinib concentration of the clear aqueous solution at 0 hour before the filtration.

TABLE 4

| | | Average |
|---|---|---|
| | Ibrutinib | Ibrutinib |
| Solution | Concentration | Concentration |
| Number | (mg/ml) | (mg/ml) |
| IB-5-3h-1 | 0.2187 | 0.2185 |
| IB-5-3h-2 | 0.2189 | |
| IB-5-3h-3 | 0.2179 | |

At 4 hours, 5 ml of the clear aqueous solution was taken out from the remaining 29 ml of the aqueous solution. The experiments were done for the 5 ml of the clear aqueous solution taken out at 4 hour using the same protocol as for the 5 ml of the clear aqueous solution taken out at 1 hour. Based on the HPLC data and the measurement data of example 17, the ibrutinib concentrations of the solution IB-5-4h have been calculated and shown in the Table 5. At 4 hours, the ibrutinib concentration of the clear aqueous solution after the filtration was about 96.70% of the ibrutinib concentration of the clear aqueous solution at 0 hour before the filtration.

TABLE 5

| | | Average |
|---|---|---|
| | Ibrutinib | Ibrutinib |
| Solution | Concentration | Concentration |
| Number | (mg/ml) | (mg/ml) |
| IB-5-4h-1 | 0.2177 | 0.2168 |
| IB-5-4h-2 | 0.2164 | |
| IB-5-4h-3 | 0.2164 | |

At 5 hours, 5 ml of the clear aqueous solution was taken out from the remaining 24 ml of the aqueous solution. The experiments were done for the 5 ml of the clear aqueous solution taken out at 5 hours using the same protocol as for the 5 ml of the clear aqueous solution taken out at 1 hour. Based on the HPLC data and the measurement data of example 17, the ibrutinib concentrations of the solution IB-5-5h have been calculated and shown in the Table 6. At 5 hour, the ibrutinib concentration of the clear aqueous solution after the filtration was about 95.32% of the ibrutinib concentration of the clear aqueous solution at 0 hour before the filtration.

TABLE 6

| | | Average |
|---|---|---|
| | Ibrutinib | Ibrutinib |
| Solution | Concentration | Concentration |
| Number | (mg/ml) | (mg/ml) |
| IB-5-5h-1 | 0.2137 | 0.2137 |
| IB-5-5h-2 | 0.2140 | |
| IB-5-5h-3 | 0.2134 | |

At 6 hours, 5 ml of the clear aqueous solution was taken out from the remaining 19 ml of the aqueous solution. The experiments were done for the 5 ml of the clear aqueous solution taken out at 6 hours using the same protocol as for the 5 ml of the clear aqueous solution taken out at 1 hour. Based on the HPLC data and the measurement data of example 17, the ibrutinib concentrations of the solution IB-5-6h have been calculated and shown in the Table 7. At 6 hours, the ibrutinib concentration of the clear aqueous solution after the filtration was about 94.42% of the ibrutinib concentration of the clear aqueous solution at 0 hour before the filtration.

TABLE 7

| | | Average |
|---|---|---|
| | Ibrutinib | Ibrutinib |
| Solution | Concentration | Concentration |
| Number | (mg/ml) | (mg/ml) |
| IB-5-6h-1 | 0.2120 | 0.2117 |
| IB-5-6h-2 | 0.2116 | |
| IB-5-6h-3 | 0.2116 | |

At 24 hours, 5 ml of the clear aqueous solution was taken out from the remaining 14 ml of the aqueous solution. The experiments were done for the 5 ml of the clear aqueous solution taken out at 24 hour using the same protocol as for the 5 ml of the clear aqueous solution taken out at 1 hour. Based on the HPLC data and the measurement data of example 17, the ibrutinib concentrations of the solution IB-5-24h have been calculated and shown in the Table 8. At 24 hour, the ibrutinib concentration of the clear aqueous solution after the filtration was about 80.24% of the ibrutinib concentration of the clear aqueous solution at 0 hour before the filtration.

TABLE 8

| | | Average |
|---|---|---|
| | Ibrutinib | Ibrutinib |
| Solution | Concentration | Concentration |
| Number | (mg/ml) | (mg/ml) |
| IB-5-24h-1 | 0.1799 | 0.1799 |
| IB-5-24h-2 | 0.1802 | |
| IB-5-24h-3 | 0.1796 | |

### Example 19: Composition Comprising Ibrutinib and Human Serum Albumin (HSA)

The Ratio by Weight of Ibrutinib to HSA Prepared was about 1:180.

Ibrutinib (15 mg) was dissolved in methanol (15.4 ml) in a vial to give a clear solution. HSA (2700 mg) (native fatty acid free human serum albumin purchased from SeraCare Life Sciences, product code: HS-455-80, which contains fatty acids <0.2 mg/gm) as a powder was dissolved in 36 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature. This aqueous solution stays clear with no precipitation after 6 hours at room temperature.

### Example 20: Composition Comprising Ibrutinib and Human Serum Albumin (HSA)

The Ratio by Weight of Ibrutinib to HSA Prepared was about 1:190.

Ibrutinib (15 mg) was dissolved in methanol (16.3 ml) in a vial to give a clear solution. HSA (2850 mg) (native fatty acid free human serum albumin purchased from SeraCare Life Sciences, product code: HS-455-80, which contains fatty acids <0.2 mg/gm) as a powder was dissolved in 38 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature. This aqueous solution stays clear with no precipitation after 6 hours at room temperature.

### Example 21: Composition Comprising Ibrutinib and Human Serum Albumin (Recombinant Human Serum Albumin)

The Ratio by Weight of Ibrutinib to HSA Prepared was about 1:200.

Ibrutinib (1 mg) was dissolved in methanol (1.7 ml) in a vial to give a clear solution. HSA (200 mg) (fatty acid free recombinant human serum albumin (no fatty acids detected) purchased from Wuhan Healthgen Biotechnology Corp., www.oryzogen.com) as a powder was dissolved in 4 ml of water in a round bottom flask. The methanol solution of ibrutinib was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature.

### Example 22: Measure the Ibrutinib Concentrations in the Aqueous Solutions Before and after the Filtration at 0 Hour, and after the Filtration at 1 Hour, 2 Hour, 3 Hour, 4 Hour, 5 Hour, 6 Hour, and 24 Hour

2.5 g of the lyophilized solid of the composition comprising ibrutinib and HSA (the ratio by weight about 1:180) from Example 19 was dissolved in 50 ml of water to give a clear aqueous solution, which was kept at about 25° C. Immediately after the lyophilized solid was dissolved in water, 6 ml of the clear aqueous solution was taken out from the 50 ml solution. Then 1 ml of the solution was taken out from the 6 ml clear aqueous solution to give the solution IB-0-0h, and the remaining 5 ml of the solution was filtered by the same 0.22 micron aqueous phase filter at 1 ml at a time to give the solutions IB-1-0h, IB-2-0h, IB-3-0h, IB-4-0h, and IB-5-0h. To 200 μl of the solutions IB-0-0h and IB-5-0h were added 800 μl of acetonitrile separately. The mixtures were vortexed for seconds and then centrifuged at 4,000 g for 5 minutes. The supernatants were removed and collected followed by injection on HPLC. The same procedure was repeated 2 more times for each of solutions IB-0-0h and IB-5-0h. Based on the HPLC data and the measurement data of example 17, the ibrutinib concentrations of the solutions of IB-0-0h, and IB-5-0h have been calculated and shown in the Table 9. At 0 hour, the ibrutinib concentration of the clear aqueous solution after the filtration was about 99.96% of the ibrutinib concentration of the clear aqueous solution before the filtration.

TABLE 9

| | | Average |
|---|---|---|
| | Ibrutinib | Ibrutinib |
| Solution | Concentration | Concentration |
| Number | (mg/ml) | (mg/ml) |
| IB-0-0h-1 | 0.2496 | 0.2502 |
| IB-0-0h-2 | 0.2500 | |
| IB-0-0h-3 | 0.2510 | |
| IB-5-0h-1 | 0.2510 | 0.2501 |
| IB-5-0h-2 | 0.2500 | |
| IB-5-0h-3 | 0.2494 | |

At 1 hour, 5 ml of the clear aqueous solution was taken out from the remaining 44 ml of the aqueous solution. Then 1 ml of the solution was taken out from the 5 ml clear aqueous solution and filtered by a 0.22 micron aqueous phase filter to give the solution IB-1-1h, and the remaining 4 ml of the solution was filtered by the same 0.22 micron aqueous phase filter at 1 ml at a time to give the solutions IB-2-1h, IB-3-1h, IB-4-1h, and IB-5-1h. To 200 μl of the solution IB-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution IB-5-1h. Based on the HPLC data and the measurement data of example 17, the ibrutinib concentrations of the solution IB-5-1h have been calculated and shown in the Table 10. At 1 hour, the ibrutinib concentration of the clear aqueous solution after the filtration was about 98.56% of the ibrutinib concentration of the clear aqueous solution at 0 hour before the filtration.

TABLE 10

| | | Average |
|---|---|---|
| | Ibrutinib | Ibrutinib |
| Solution | Concentration | Concentration |
| Number | (mg/ml) | (mg/ml) |
| IB-5-1h-1 | 0.2471 | 0.2466 |
| IB-5-1h-2 | 0.2467 | |
| IB-5-1h-3 | 0.2461 | |

At 2 hours, 5 ml of the clear aqueous solution was taken out from the remaining 39 ml of the aqueous solution. The experiments were done for the 5 ml of the clear aqueous solution taken out at 2 hours using the same protocol as for the 5 ml of the clear aqueous solution taken out at 1 hour. Based on the HPLC data and the measurement data of example 17, the ibrutinib concentrations of the solution IB-5-2h have been calculated and shown in the Table 11. At 2 hours, the ibrutinib concentration of the clear aqueous solution after the filtration was about 97.72% of the ibrutinib concentration of the clear aqueous solution at 0 hour before the filtration.

TABLE 11

| | | Average |
|---|---|---|
| | Ibrutinib | Ibrutinib |
| Solution | Concentration | Concentration |
| Number | (mg/ml) | (mg/ml) |
| IB-5-2h-1 | 0.2442 | 0.2445 |
| IB-5-2h-2 | 0.2446 | |
| IB-5-2h-3 | 0.2447 | |

At 3 hours, 5 ml of the clear aqueous solution was taken out from the remaining 34 ml of the aqueous solution. The experiments were done for the 5 ml of the clear aqueous solution taken out at 3 hour using the same protocol as for the 5 ml of the clear aqueous solution taken out at 1 hour. Based on the HPLC data and the measurement data of example 17, the ibrutinib concentrations of the solution IB-5-3h have been calculated and shown in the Table 12. At 3 hours, the ibrutinib concentration of the clear aqueous solution after the filtration was about 96.76% of the ibrutinib concentration of the clear aqueous solution at 0 hour before the filtration.

TABLE 12

| | | Average |
|---|---|---|
| | Ibrutinib | Ibrutinib |
| Solution | Concentration | Concentration |
| Number | (mg/ml) | (mg/ml) |
| IB-5-3h-1 | 0.2419 | 0.2421 |
| IB-5-3h-2 | 0.2427 | |
| IB-5-3h-3 | 0.2418 | |

At 4 hours, 5 ml of the clear aqueous solution was taken out from the remaining 29 ml of the aqueous solution. The experiments were done for the 5 ml of the clear aqueous solution taken out at 4 hour using the same protocol as for the 5 ml of the clear aqueous solution taken out at 1 hour. Based on the HPLC data and the measurement data of example 17, the ibrutinib concentrations of the solution IB-5-4h have been calculated and shown in the Table 13. At 4 hours, the ibrutinib concentration of the clear aqueous solution after the filtration was about 96.16% of the ibrutinib concentration of the clear aqueous solution at 0 hour before the filtration.

TABLE 13

| | | Average |
|---|---|---|
| | Ibrutinib | Ibrutinib |
| Solution | Concentration | Concentration |
| Number | (mg/ml) | (mg/ml) |
| IB-5-4h-1 | 0.2411 | 0.2406 |
| IB-5-4h-2 | 0.2405 | |
| IB-5-4h-3 | 0.2403 | |

At 5 hours, 5 ml of the clear aqueous solution was taken out from the remaining 24 ml of the aqueous solution. The experiments were done for the 5 ml of the clear aqueous solution taken out at 5 hours using the same protocol as for the 5 ml of the clear aqueous solution taken out at 1 hour. Based on the HPLC data and the measurement data of example 17, the ibrutinib concentrations of the solution IB-5-5h have been calculated and shown in the Table 14. At 5 hour, the ibrutinib concentration of the clear aqueous solution after the filtration was about 95.60% of the ibrutinib concentration of the clear aqueous solution at 0 hour before the filtration.

TABLE 14

| | | Average |
|---|---|---|
| | Ibrutinib | Ibrutinib |
| Solution | Concentration | Concentration |
| Number | (mg/ml) | (mg/ml) |
| IB-5-5h-1 | 0.2390 | 0.2392 |
| IB-5-5h-2 | 0.2400 | |
| IB-5-5h-3 | 0.2386 | |

At 6 hours, 5 ml of the clear aqueous solution was taken out from the remaining 19 ml of the aqueous solution. The experiments were done for the 5 ml of the clear aqueous solution taken out at 6 hours using the same protocol as for the 5 ml of the clear aqueous solution taken out at 1 hour. Based on the HPLC data and the measurement data of example 17, the ibrutinib concentrations of the solution IB-5-6h have been calculated and shown in the Table 15. At 6 hours, the ibrutinib concentration of the clear aqueous solution after the filtration was about 95.00% of the ibrutinib concentration of the clear aqueous solution at 0 hour before the filtration.

TABLE 15

| | | Average |
|---|---|---|
| | Ibrutinib | Ibrutinib |
| Solution | Concentration | Concentration |
| Number | (mg/ml) | (mg/ml) |
| IB-5-6h-1 | 0.2369 | 0.2377 |
| IB-5-6h-2 | 0.2378 | |
| IB-5-6h-3 | 0.2383 | |

At 24 hours, 5 ml of the clear aqueous solution was taken out from the remaining 14 ml of the aqueous solution. The experiments were done for the 5 ml of the clear aqueous solution taken out at 24 hour using the same protocol as for the 5 ml of the clear aqueous solution taken out at 1 hour. Based on the HPLC data and the measurement data of example 17, the ibrutinib concentrations of the solution IB-5-24h have been calculated and shown in the Table 16. At 24 hour, the ibrutinib concentration of the clear aqueous solution after the filtration was about 82.89% of the ibrutinib concentration of the clear aqueous solution at 0 hour before the filtration.

TABLE 16

| | | Average |
|---|---|---|
| | Ibrutinib | Ibrutinib |
| Solution | Concentration | Concentration |
| Number | (mg/ml) | (mg/ml) |
| IB-5-24h-1 | 0.2067 | 0.2074 |
| IB-5-24h-2 | 0.2075 | |
| IB-5-24h-3 | 0.2081 | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-72. (canceled)

73. A composition comprising a non-covalently bound complex comprising ibrutinib and human serum albumin, wherein the ibrutinib and the human serum albumin in the composition have a ratio by weight from about 1:5 to about 1:2000.

74. The composition of claim 73, wherein the ibrutinib and the human serum albumin in the composition have a ratio by weight from about 1:100 to about 1:1000.

75. The composition of claim 73, wherein the human serum albumin is essentially fatty acid free.

76. A composition comprising ibrutinib and human serum albumin, wherein the ibrutinib and the human serum albumin in the composition have a ratio by weight from about 1:5 to about 1:2000.

77. The composition of claim 76, wherein the ibrutinib and the human serum albumin in the composition have a ratio by weight from about 1:100 to about 1:1000.

78. The composition of claim 76, wherein the human serum albumin is a native human serum albumin.

79. The composition of claim 76, wherein the human serum albumin is a recombinant human serum albumin.

80. The composition of claim 76, wherein the composition is a solid formulation.

81. The composition of claim 76, wherein the composition is an aqueous formulation.

82. A pharmaceutical composition comprising the composition of claim 76, and a pharmaceutically acceptable carrier.

83. A method of treating a cancer, the method comprising the step of administering to a subject in need thereof a therapeutically effective amount of a pharmaceutical composition of claim 82.

84. The composition of claim 76, wherein the composition is produced by a method comprising the steps of:
(i) obtaining an organic solution of ibrutinib in a polar water-miscible organic solvent;
(ii) obtaining a first aqueous solution of human serum albumin; and
(iii) mixing the organic solution of ibrutinib and the first aqueous solution of human serum albumin to obtain a second aqueous solution comprising the composition comprising ibrutinib and human serum albumin.

85. The composition of claim 84, wherein the human serum albumin is a native human serum albumin.

86. The composition of claim 84, wherein the human serum albumin is a recombinant human serum albumin.

87. The composition of claim 84, wherein the amount of aqueous solvent in the first aqueous solution is from about 0.01 mL to about 0.05 mL per 1 mg of human serum albumin.

88. The composition of claim 84, wherein the polar water-miscible organic solvent is an alcohol selected from the group consisting of ethanol, isopropanol, n-butanol, and mixtures thereof.

89. The composition of claim 84, wherein the mixing comprises adding the organic solution to the first aqueous solution.

90. The composition of claim 84, further comprising removing the organic solvent and the aqueous solvent from the second aqueous solution to obtain the composition comprising ibrutinib and human serum albumin.

91. A pharmaceutical composition comprising the composition of claim 84, and a pharmaceutically acceptable carrier.

92. A method of treating a cancer, the method comprising the step of administering to a subject in need thereof a therapeutically effective amount of a pharmaceutical composition of claim 91.
